(19) United States
(12) Patent Application Publication
(10) Pub. No.: US 2021/0236554 A1
(43) Pub. Date: Aug. 5, 2021

(54) LOW DOSE RADIATION CONDITIONING FOR IMMUNOTHERAPY

(71) Applicant: MEMORIAL SLOAN-KETTERING CANCER CENTER, New York, NY (US)

(72) Inventors: Michel Sadelain, New York, NY (US); Carl DeSelm, St. Louis, MO (US)

(73) Assignee: MEMORIAL SLOAN-KETTERING CANCER CENTER, New York, NY (US)

(21) Appl. No.: 17/233,924

(22) Filed: Apr. 19, 2021

Related U.S. Application Data
(63) Continuation of application No. PCT/US2019/057004, filed on Oct. 18, 2019.
(60) Provisional application No. 62/748,171, filed on Oct. 19, 2018.

(51) Int. Cl. A61K 35/17; C07K 14/705; C07K 14/725; A61P 35/02; A61P 35/00; A61N 5/10

(52) U.S. Cl. CPC A61K 35/17; C07K 14/70575; C07K 14/7051; A61N 2005/1021; A61P 35/00; A61N 5/1001; A61N 5/1077; A61P 35/02

(57) ABSTRACT

The present disclosure provides methods and compositions for treating cancers and pathogens. It relates to an immunoresponsive cell comprising an antigen-recognizing receptor (e.g., a chimeric antigen receptor (CAR) or a T cell receptor (TCR)), and expressing a secretable TRAIL polypeptide.

### CROSS-REFERENCE TO RELATED APPLICATIONS

This application is a Continuation of International Patent Application No. PCT/US19/57004, filed on Oct. 18, 2019, which claims priority to U.S. Provisional Application No. 62/748,171, filed on Oct. 19, 2018, the content of each of which are incorporated by reference in their entirety, and to each of which priority is claimed.

### SEQUENCE LISTING

The specification further incorporates by reference the Sequence Listing submitted herewith via EFS on Apr. 19, 2021. Pursuant to 37 C.F.R. § 1.52(e)(5), the Sequence Listing text file, identified as 0727341250 SL.txt, is 50,566 bytes and was created on Apr. 19, 2021. The Sequence Listing electronically filed herewith, does not extend beyond the scope of the specification and thus does not contain new matter.

### INTRODUCTION

The presently disclosed subject matter provides methods and compositions for enhancing the immune response toward cancers and pathogens. It relates to immunoresponsive cells comprising antigen-recognizing receptors (e.g., chimeric antigen receptors (CARs) or T cell receptors (TCRs)) that are engineered to express a TRAIL polypeptide. These engineered immunoresponsive cells are antigen-directed, promote recruitment of other cytokines and exhibit enhanced anti-target efficacy. It also relates to methods of adoptive cell therapy combined with low dose radiation therapy (RT).

### BACKGROUND OF THE INVENTION

CD19 CAR T cells achieve a complete response (CR) in a majority of patients with refractory, relapsed B cell malignancies (Dunbar et al., Science (2018); 359). Responses to CAR therapy targeting solid tumors have to date been relatively scarce (Sadelain et al., Nature (2017); 545:423-431). One of the challenges to overcome in all cancers and especially solid tumors is antigen heterogeneity. Whereas all or most B cell malignancies express CD19 (Brentjens et al., Nat Med (2003); 9:279-286), numerous potential CAR targets are only expressed in a fraction of all tumor cells within a patient, posing the risk of antigen escape. Low-level antigen expression may also result in resistance to CAR therapy (Fry et al., Nat Med (2018); 24:20-28). Targeting two or more antigens can be implemented in the event of a defined escape population or clone (Wilkie et al., J Clin Immunol (2012); 32:1059-1070; Kloss et al., Nat Biotechnol (2013); 31:71-75; Ruella et al., J Clin Invest (2016); 126:3814-3826; Hegde et al., J Clin Invest (2016); 126:3036-3052; Zah et al., Cancer Immunol Res 2016; 4:498-508), but other approaches are needed to overcome greater or undefined target heterogeneity. Accordingly, there are needs for novel therapeutic strategies to design CARs targeting antigens that are highly expressed in solid tumor cells and for strategies capable of inducing potent cancer eradication with minimal toxicity and immunogenicity.

### SUMMARY OF THE INVENTION

The presently disclosed subject matter provides immunoresponsive cells (e.g., T cells, Tumor Infiltrating Lymphocytes, Natural Killer (NK) cells, cytotoxic T lymphocytes (CTLs), Natural Killer T (NK-T) cells or regulatory T cells) comprising: (a) an antigen-recognizing receptor that binds to an antigen, and (b) an exogenous TRAIL polypeptide.

The presently disclosed subject matter also provides immunoresponsive cells comprising: (a) an antigen-recognizing receptor that binds to an antigen, and (b) a modified enhancer or promoter at an endogenous TRAIL gene locus.

In certain embodiments, the modified enhancer or promoter enhances gene expression of the endogenous TRAIL gene. In certain embodiments, the modification comprises replacement of an endogenous promoter with a constitutive promoter or an inducible promoter, or insertion of a constitutive promoter or inducible promoter to the promoter region of the endogenous TRAIL gene locus. In certain embodiments, the constitutive promoter is selected from the group consisting of a CMV promoter, an EF1a promoter, a SV40 promoter, a PGK1 promoter, a Ubc promoter, a beta-actin promoter, and a CAG promoter. In certain embodiments, the inducible promoter is selected from the group consisting of a tetracycline response element (TRE) promoter and an estrogen response element (ERE) promoter.

In certain embodiments, the antigen is a tumor antigen or a pathogen antigen. In certain embodiments, the exogenous TRAIL polypeptide is secreted. In certain embodiments, the antigen-recognizing receptor is a T cell receptor (TCR), a chimeric antigen receptor (CAR) or a truncated CAR. In certain embodiments, the antigen recognizing receptor is exogenous or endogenous. In certain embodiments, the antigen recognizing receptor is recombinantly expressed. In certain embodiments, the antigen-recognizing receptor is expressed from a vector.

In certain embodiments, the exogenous TRAIL polypeptide is expressed from a vector.

In certain embodiments, the cell is selected from the group consisting of a T cell, a Natural Killer (NK) cell, a cytotoxic T lymphocyte (CTL), a regulatory T cell, a Natural Killer T (NKT) cell, a human embryonic stem cell, and a pluripotent stem cell from which lymphoid cells may be differentiated. In certain embodiments, the immunoresponsive cell is autologous or allogeneic to the intended recipient.

In certain embodiments, the antigen is a tumor antigen. In certain embodiments, the tumor antigen is selected from the group consisting of CD19, Sialyl Lewis A, MUC16, MUC1, CAlX, CEA, CD8, CD7, CD10, CD20, CD22, CD30, CD33, CLL1 CD34, CD38, CD41, CD44, CD49f, CD56, CD74, CD133, CD138, a cytomegalovirus (CMV) infected cell antigen, EGP-2, EGP-40, EpCAM, erb-B2,3,4, FBP, Fetal acetylcholine receptor, folate receptor-α, GD2, GD3, HER-2, hTERT, IL-13R-a2, κ-light chain, KDR, LeY, L1 cell adhesion molecule, MAGE-A1, Mesothelin, ERBB2, MAGEA3, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, ERBB, ITGB5, PTPRJ, SLC30A1, EMC10, SLC6A6, TNFRSF1B, CD82, ITGAX, CR1, DAGLB, SEMA4A, TLR2, LTB4R, P2RY13, LILRB2, EMB, CD96, LILRB3, LILRA6, LILRA2, ADGRE2, LILRB4, CD70, CCR1, CCR4, TACI, TRBC1, and TRBC2. In certain embodiments, the antigen is CD19. In certain embodiments, the antigen is Sialyl Lewis A.

In certain embodiments, the TRAIL polypeptide comprises a heterologous signal sequence at the amino-terminus. In certain embodiments, the antigen-recognizing receptor is a CAR. In certain embodiments, the CAR comprises an extracellular antigen-binding domain, a transmembrane domain, and an intracellular signaling domain. In certain embodiments, the CAR is 1928z. In certain embodiments, the TRAIL polypeptide comprises an amino acid sequence that is at least about 80% homologous or identical to the amino acid sequence set forth in SEQ ID NO: 63, or a fragment thereof. In certain embodiments, the TRAIL polypeptide has the amino acid sequence set forth in SEQ ID NO: 63, or a fragment thereof.

The presently disclosed subject matter provides compositions (e.g., pharmaceutical compositions) comprising an effective amount of an immunoresponsive cells disclosed herein. In certain embodiments, the composition is a pharmaceutical composition that further comprises a pharmaceutically acceptable excipient. In certain embodiments, the pharmaceutical composition is for treating a neoplasia.

The presently disclosed subject matter also provides methods of reducing tumor burden in a subject. In certain embodiments, the method comprises administering to the subject an effective amount of immunoresponsive cells disclosed herein or a composition comprising thereof. In certain embodiments, the method reduces the number of tumor cells. In certain embodiments, the method reduces tumor size. In certain embodiments, the method eradicates the tumor in the subject.

The presently disclosed subject matter also provides methods of treating and/or preventing a neoplasia. In certain embodiments, the method comprises administering to the subject an effective amount of immunoresponsive cells disclosed herein or a composition comprising thereof.

The presently disclosed subject matter also provides methods of lengthening survival of a subject having a neoplasia. In certain embodiments, the method comprises administering to the subject an effective amount of immunoresponsive cells disclosed herein or a composition comprising thereof.

In certain embodiments, the tumor and/or neoplasia is selected from the group consisting of blood cancer, B cell leukemia, multiple myeloma, lymphoblastic leukemia (ALL), chronic lymphocytic leukemia, non-Hodgkin's lymphoma, and pancreatic cancer. In certain embodiments, the neoplasia is B cell leukemia, multiple myeloma, lymphoblastic leukemia (ALL), chronic lymphocytic leukemia, or non-Hodgkin's lymphoma, and the antigen is CD19. In certain embodiments, the neoplasia is pancreatic cancer, and the antigen is Sialyl Lewis A.

The presently disclosed subject matter provides methods of treating blood cancer in a subject in need thereof. In certain embodiments, the method comprises administering to the subject an effective amount of T cells, wherein the T cell comprises: (a) an antigen-recognizing receptor that binds to CD19, and an exogenous TRAIL polypeptide; or (b) an antigen-recognizing receptor that binds to CD19, and a modified enhancer or promoter at an endogenous TRAIL gene locus. In certain embodiments, the blood cancer is selected from the group consisting of B cell leukemia, multiple myeloma, acute lymphoblastic leukemia (ALL), chronic lymphocytic leukemia, and non-Hodgkin's lymphoma.

The presently disclosed subject matter also provides methods of treating pancreatic cancer in a subject in need thereof. In certain embodiments, the method comprises administering to the subject an effective amount of T cells, wherein the T cell comprises: (a) an antigen-recognizing receptor that binds to Sialyl Lewis A, and an exogenous TRAIL polypeptide; or (b) an antigen-recognizing receptor that binds to Sialyl Lewis A, and a modified enhancer or promoter at an endogenous TRAIL gene locus.

In certain embodiments, the method further comprises administering a radiation therapy to the subject. In certain embodiments, the radiation therapy is administered prior to the administration of the immunoresponsive cells or the composition.

The presently disclosed subject matter provides methods for producing an antigen-specific immunoresponsive cell. In certain embodiments, the method comprises introducing into an immunoresponsive cell (a) a first nucleic acid sequence encoding an antigen-recognizing receptor that binds to an antigen; and (b) a second nucleic sequence encoding an exogenous TRAIL polypeptide, wherein each of the first and second nucleic acid sequence optionally operably linked to a promoter element. In certain embodiments, one or both of the first and second nucleic acid sequences are comprised in a vector. In certain embodiments, the vector is a retroviral vector.

The presently disclosed subject matter also provides nucleic acid compositions comprising (a) a first nucleic acid sequence encoding an antigen-recognizing receptor and (b) a second nucleic acid sequence encoding an exogenous TRAIL polypeptide, each optionally operably linked to a promoter element. In certain embodiments, one or both of the first and second nucleic acid sequences are comprised in a vector. In certain embodiments, the vector is a retroviral vector.

The presently disclosed subject matter provides vector comprising a nucleic acid composition disclosed herein.

The presently disclosed subject matter provides kit comprising an immunoresponsive cell, a pharmaceutical composition, a nucleic acid composition, or a vector disclosed herein. In certain embodiments, the kit further comprises written instructions for treating and/or preventing a neoplasm or a pathogen infection.

The presently disclosed subject matter provides methods of reducing tumor burden or treating and/or preventing a neoplasia in a subject, the method comprising: i) administering to the subject a radiation therapy; and ii) administering to the subject an effective amount of immunoresponsive cells or a pharmaceutical composition comprising thereof.

In certain embodiments, the radiation therapy is administered in a total dose of no more than about 30 Gy. In certain embodiments, the radiation therapy is administered in a total dose of between about 1 Gy and about 20 Gy. In certain embodiments, the radiation therapy is administered no later than about 20 days prior to the administration of the immunoresponsive cells. In certain embodiments, the radiation therapy is administered no later than about 10 days prior to the administration of the immunoresponsive cells. In certain embodiments, the radiation therapy is an external beam radiation therapy, a brachytherapy or a systemic radioisotope therapy. In certain embodiments, the immunoresponsive cell comprises an antigen recognizing receptor. In certain embodiments, the immunoresponsive cell is a immunoresponsive cell disclosed herein.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1G depict that radiation therapy (RT) sensitizes Nalm6 ALL to CAR T cell killing, in vitro and in vivo. FIGS. 1A-1D show that mice with established Nalm6 were treated with RT followed by CAR T cells (n≥5 mice/group, repeated twice). FIG. 1C shows that “RT (mouse pre-tumor)” mice received RT before tumor injection. FIG. 1E shows that two days after RT or no RT, rather than treating with CAR T cells mice were euthanized, femoral bones harvested, and tumor cells quantified by flow cytometry. FIG. 1F shows that CTLs in which Nalm6 tumor cells were exposed to RT or no RT 48 hours prior to T cells. FIG. 1G shows that CD19 levels on Nalm6 were unchanged following RT. All RT doses to Nalm6 are 1.8 Gy, unless specifically specified as 0.5 Gy.

FIGS. 2A-2E depict that DR5 is induced on the tumor by low-dose RT, and is important for RT sensitization to CAR T cell killing. FIG. 2A shows DR5 mRNA quantified by qPCR 6 hours post exposure to RT, and surface protein expression 48 hours post exposure to RT. FIG. 2B depicts TRAIL mRNA expression and protein levels in the media of 1928z CAR T cells after exposure to target antigen (CD19-expressing 3T3 cells). FIG. 2C shows TRAIL protein was quantified in the media of 1928z and 19(del) CAR T cells grown on target cells expressing or not expressing the CD19 target antigen. FIGS. 2D-2E show Overall Survival (FIG. 2D) and tumor burden (FIG. 2E) of mice harboring DR5^{-/-} or DR5^{wt} Nalm6 tumor, treated as in FIG. 1A (≥5 mice/group).

FIGS. 3A-3C depict RT and CAR T timing affects efficacy. FIG. 3A shows time course of DR5 expression on Nalm6 after RT. FIGS. 3B and 3C show OS (FIG. 3B) and tumor burden (FIG. 3C) of Nalm6-bearing, RT sensitized mice given CAR T cells 1 or 7 days following RT (≥5 mice/group).

FIGS. 4A-4D depict that RT sensitizes pancreatic cancer to CAR T cell killing without affecting target antigen expression, in vivo and in vitro. FIG. 4A illustrates Capan2 pancreatic cancer cells were exposed to low dose RT (2 Gy for all pancreatic cancer studies), and 48 hours later incubated with CAR T cells at indicated ratios for 18 hours, after which percent killing was determined. FIG. 4B depicts target antigen expression levels were unchanged 48 hours after RT. FIG. 4C shows orthotopic mouse model, in which PDAC cells are injected into the pancreas, grow for 9 days, then are treated with different doses of CAR T cells, or RT+CAR T cells. FIG. 4D illustrates Tumor burden assessed by bioluminescence imaging over time.

FIGS. 5A-5D depict that TRAIL expressed by activated CAR T cells is functionally significant against antigen-negative tumor cells in a heterogeneous tumor population exposed to low-dose radiation. FIG. 5A shows CAR-activated T cells produce TRAIL, which acts upon radiation-sensitized antigen-positive and antigen-negative tumor cells. Capan2 PDAC cells were sorted into Ag⁺ and luciferase-expressing Ag⁻ populations, and mixed at a ratio of 75:25 Ag⁺ Ag⁻. FIGS. 5B-5C show tumor cells exposed to low-dose RT, and cocultured with the indicated CAR T cells for four days, followed by luciferase-based quantification of Ag⁻ cell killing. FIG. 5D shows RT-sensitized Ag⁻ cells labeled with CTV mixed with unlabeled Ag⁺ cells and quantified 6 days after TRAIL^{-/-} or TRAIL^{wt} CART cell coculture. **p<0.01.

FIGS. 6A-6C depict that sensitizing RT transcriptionally primes pancreatic cancer cells for TRAIL-induced death. FIG. 6A shows RNA expression levels of signaling molecules known to mediate various TRAIL responses, including survival and migration, tumor-supportive inflammation, necroptosis, apoptosis, and death receptor endocytosis, quantified by RNAseq before and after RT exposure to Capan2 pancreatic cancer cells in three biologic replicates. Significantly induced and downregulated molecules are shown in red and green, respectively, with magnitude represented by color gradient. Molecules in gray were not significantly changed. FIG. 6B shows GSA of pathways implicated in Akt signaling, NFkB signaling, and cell death significantly perturbed by low-dose RT (FDR<0.05), charted by mean LFC (log 2[fold change]). FIG. 6C shows CTV-labeled Ag⁻ cells were exposed to RT two days before coculture with unlabeled Ag+ cells, annexin-V 595, and TRAIL^{-/-} or TRAIL^{wt} CAR T cells. Cultures were monitored by live video microscopy and Ag⁻ cell apoptosis quantified over time.

FIGS. 7A-7M depict that sensitizing RT allows CAR T cells to eliminate heterogeneous PDAC in vivo. FIG. 7A shows Capan2 tumor cells mixed at 75:25 LeA(+):(−), then injected into the pancreas of NSG mice. After tumor established for 9 days, mice were given RT, followed by CAR T cells. FIG. 7B shows waterfall plot of tumor volume change at time of death among different treatment groups. In FIGS. 7C-7H, BLI was performed weekly. As shown in FIG. 7I, T cell infiltration of tumor from CAR or RT+CAR treated mice was determined using BLI T cell imaging (detecting G-Luc on the transduced T cell) over the first 19 days, and by IHC from mice sacrificed on day 21 (J-K, all ns). FIG. 7L shows tumors in mice that progressed display reduced target antigen expression over time by FACS. FIG. 7M shows BLI of mice treated with RT+L(del) or RT+L(del)-TRAIL CAR T cells.

FIGS. 8A-8H depict that outcome of patient with heterogeneous tumor treated with palliative RT and CAR T cells. FIG. 8A shows total body or local RT delivered to mice harboring heterogeneous tumor of the pancreas using image-guided radiation, followed by CAR T cells. FIG. 8B shows tumor burden monitored by BLI. FIGS. 8C-8E depict patient biopsy before CAR T cell treatment examined for CD19 by IHC (FIG. 8C) and flow cytometry (FIG. 8D). FIGS. 8F-8G illustrate patient tumor biopsy irradiated with 0 or 4 Gy×5 ex vivo and assessed for DR5 surface expression. RT induced DR5 on the bulk tumor sample, and specifically the surviving antigen-negative tumor cells (FIG. 8G). FIG. 8H depicts FDG-PET scan before, and 1, 2, and 6 months post palliative leg RT and systemic 1928z CAR T cells.

FIGS. 9A-9C depict that very low doses of RT and CAR T cells can eliminate Nalm6 ALL in vivo. FIGS. 9A-9C depict mice were treated with 0.5 Gy followed by 10⁴ 1928 z CAR T cells or control L28z CAR T cells the following day, four days after Nalm6 ALL injection. Overall survival and tumor burden, measured by BLI, are shown.

FIG. 10 depicts that DR5 is induced in response to low dose RT. Nalm6 were irradiated at 0.5 Gy in vitro and RNA harvested 6 hours later. Putative surface protein products significantly induced >2 fold were identified and listed.

FIG. 11 depicts that GSA identifies pathways significantly altered (FDR<0.05) by low dose RT. FDR=false discovery rate. LFC=log 2 fold change.

FIGS. 12A-12C depict that Surface protein expression of candidates identified by RNAseq. FIG. 12A shows indicated proteins examined by FACS 48 hours after 0, 0.5, or 1.8 Gy to Nalm6 in vitro. FIG. 12B shows mice with established Nalm6 given 0 or 1.8 Gy RT, and 48 hours later euthanized and examined for DR5 levels on the tumor cells, showing induction in vivo. FIG. 12C illustrates that DR5 was knocked out in Nalm6 tumor cells using CRISPR, and sorted by flow cytometry. Two weeks post sort 97% of cells remained DR5^{-/-} and did not induce DR5 after RT.

FIGS. 13A-13C depict that CAR targeting LeA specifically lyses cells that express LeA. FIG. 13A shows LBBz CAR T cell design, containing membrane-bound G-Luc for imaging. FIG. 13B shows endogenous LeA expression on PC3, Capan2, and BxPC3 cells examined by flow cytometry. FIG. 13C illustrates PC3, Capan2, or BxPC3 cells mixed with LBBz or L28z CAR or untransduced T cells at various effector:target ratios for 18 hours followed by quantification of target cell killing.

FIGS. 14A-14C depict that TRAIL is expressed in response to CAR signaling, and TRAIL-knockout impairs Ag⁻ tumor cell killing after RT. FIG. 14A shows TRAIL knocked out in primary T cells using CRISPR, followed by CAR transduction. Three days after transduction TRAIL-negative cells were negatively selected on a magnetic column, followed by exposure to target cells for two days. TRAIL mRNA was quantified in TRAIL^{wt} and TRAIL^{-/-} CAR T cells after target antigen exposure (Stim), and expressed as fold change relative to TRAIL^{wt} CAR T cells without target antigen exposure. FIG. 14B shows microscopy of CTV-labeled, RT-stimulated LeA⁻ tumor cells (white) in coculture with TRAIL^{wt} or TRAIL^{-/-} LBBz CAR T cells increased LeA⁻ tumor cells after TRAIL^{-/-} CAR T cell treatment, but elimination of LeA⁻ cells after TRAIL^{wt} CAR T cell treatment. FIG. 14C illustrates a table of molecules (shown schematically in FIG. 6) known to mediate various TRAIL responses, including survival and migration, tumor-supportive inflammation, necroptosis, apoptosis, and death receptor endocytosis. Molecules with an adjusted p-value <0.05 are shown.

FIGS. 15A and 15B depict profile of tumor and T cells. FIG. 15A shows Capan2 cells FACS sorted into LeA⁺ and LeA⁻ populations, then mixed at a ratio of 75:25 LeA+/− tumor cells. LeA⁻ sorted Capan2 cells remain LeA⁻ over time. FIG. 15B shows typical T cell profile after CAR transduction and TCR knockout, before in vivo injection.

FIGS. 16A-16D depict that CAR T cells persist in vivo, penetrate tumor, and deplete Ag⁺ tumor cells in mice harboring heterogeneous Ag+/− pancreatic cancer. FIG. 16A shows cells isolated from blood, spleen, and tumor from mice treated with CAR T cells 6 weeks prior analysis for CAR T cells content. FIG. 16B shows pure T cell population controls. FIG. 16C shows IHC for CD3, CD4 and CD8 T cell penetration of 75:25 LeA+/− tumor treated LeA CART cells. FIG. 16D shows CAR T cell tumor infiltration, quantified by CTZ T-cell bioluminescent imaging over time, shows both TRAIL-knockout LBBz and L(del) CAR T cells accumulate in the pancreatic tumors over time

FIG. 17 depicts that T cell accumulation in the tumor of mice treated with total body or local RT. LBBz CAR T cells were quantified in the pancreatic tumor using bioluminescence imaging over time in mice treated with local, total body (TBI), or no RT followed by CAR T cells (FIG. 8A).

FIG. 18 depicts that DR5 surface expression following various clinically relevant, non-ablative RT dose regimens to various PDX tumors. PDX tumor cells were treated with the indicated RT dose and assessed for DR5 expression by FACS 48 hours after treatment. Two tumors (top) increased DR5 after 2 Gy, three tumors (middle row) increased DR5 after 4 Gy×5, and one tumor (bottom) was relatively unresponsive to any dose of RT.

### DETAILED DESCRIPTION OF THE INVENTION

The presently disclosed subject matter provides cells, including genetically modified immunoresponsive cells (e.g., T cells, NK cells, or CTL cells) comprising a combination of an antigen-recognizing receptor (e.g., TCR or CAR) and a secretable TRAIL polypeptide (e.g., an exogenous TRAIL polypeptide, or a nucleic acid encoding a TRAIL polypeptide). The presently disclosed subject matter also provides methods of adoptive cell therapy combined with low dose radiation therapy (RT). The presently disclosed subject matter is based, at least in part, on the discovery that low dose RT preconditioning enhances tumor killing effects of an immunoresponsive cell in subjects receiving an immunotherapy (e.g., CAR-T cells), and that a secretable TRAIL polypeptide enhances tumor killing effects of an immunoresponsive cell in subjects receiving an immunotherapy (e.g., CAR-T cells).

### 1. DEFINITIONS

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-lBB), OX40, ICOS, and MyD88. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen. Non-limiting examples of antigen-recognizing receptors include native or endogenous T cell receptors (“TCRs”), and chimeric antigen receptors (“CARs”).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}:: V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” include any nucleic acid molecule that encodes a polypeptide of interest (e.g., a TRAIL polypeptide) or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, conservative substitutions include substitutions within the following groups: glycine, alanine; valine, isoleucine, leucine; aspartic acid, glutamic acid, asparagine, glutamine; serine, threonine; lysine, arginine; and phenylalanine, tyrosine. In certain embodiments, one or more amino acid residues within or outside a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence outside a CDR region or a CDR region are altered.

As used herein, the percent homology between two amino acid sequences is equivalent to the percent identity between the two sequences. The percent identity between the two sequences is a function of the number of identical positions shared by the sequences (i.e., % homology=#of identical positions/total #of positions ×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used.

Furthermore, sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasia, and pathogen infection of cell.

An “effective amount” (or, “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered. By “enforcing tolerance” is meant preventing the activity of self-reactive cells or immunoresponsive cells that target transplanted organs or tissues.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” or “enhance” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state.

“Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises a sequence set forth in GGGGSGGGGSGGGGS [SEQ ID NO: 1].

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [SEQ ID NO: 2] (human), MYSMQLASCVTLTLVLLVNS [SEQ ID NO: 3] (mouse); the kappa leader sequence: METPAQLLFLLLLWLPDTTG [SEQ ID NO: 4] (human), METDTLLLWVLLLWVPGSTG [SEQ ID NO: 5] (mouse); the CD8 leader sequence: MALPVTALLLPLALLLHAARP [SEQ ID NO: 6] (human); the truncated human CD8 signal peptide: MALPVTALLLPLALLLHA [SEQ ID NO: 7] (human); the albumin signal sequence: MKWVTFISLLFSSAYS [SEQ ID NO: 8] (human); and the prolactin signal sequence: MDSKGSSQKGSRLLLLLVVSNLLLCQGVVS [SEQ ID NO: 9] (human).

By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen recognizing receptor (e.g., CD19, Sialyl Lewis A) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 2. ANTIGEN-RECOGNIZING RECEPTORS

The presently disclosed cells comprise an antigen-recognizing receptors that bind to an antigen of interest. In certain embodiments, the antigen-recognizing receptor is a chimeric antigen receptor (CAR). In certain embodiments, the antigen-recognizing receptor is a truncated CAR. In certain embodiments, the antigen-recognizing receptor is a T-cell receptor (TCR). The antigen-recognizing receptor can bind to a tumor antigen or a pathogen antigen.

### 2.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include CD19, Sialyl Lewis A (sLeA) carbonic anhydrase IX (CAlX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, ERBB, ITGB5, PTPRJ, SLC30A1, EMC10, SLC6A6, TNFRSF1B, CD82, ITGAX, CR1, DAGLB, SEMA4A, TLR2, LTB4R, P2RY13, LILRB2, EMB, CD96, LILRB3, LILRA6, LILRA2, ADGRE2, LILRB4, CD70, CCR1, CCR4, TACI, TRBC1, and TRBC2.

In certain embodiments, the antigen-recognizing receptor binds to CD19.

In certain embodiments, the antigen-recognizing receptor binds to a murine CD19 polypeptide. In certain embodiments, the antigen-recognizing receptor binds to a human CD19 polypeptide. In certain embodiments, the antigen-recognizing receptor binds to exon 2 of CD19.

In certain embodiments, the antigen-recognizing receptor binds to Sialyl Lewis A. Sialyl Lewis A (also known as Le^{A}, sialyl Le^{A} and SLe^{A}, CAS No. 92448-22-1) is a tetrasaccharide comprising the sugar sequence of NeuAc(a2-3)Gal(b1-3)[Fuc(a1-4)]GlcNAc. In certain embodiments, Le^{A} comprises a formula of

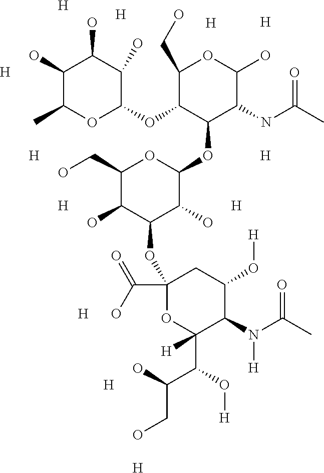

Le^{A} is present on the surface of certain cells and is involved in cell-to-cell recognition processes. It is a surface antigen expressed on 75-90% of pancreatic tumors, whereas its expression on normal human tissues is relatively low.

In certain embodiments, the antigen-recognizing receptor binds to a pathogen antigen, e.g., for use in treating and/or preventing a pathogen infection or other infectious disease, for example, in an immunocompromised subject. Non-limiting examples of pathogen includes a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtherias, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 2.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3ζ chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 2.3. Chimeric Antigen Receptor (CAR)

In certain embodiments, the antigen-recognizing receptor is a CAR. CARs are engineered receptors, which graft or confer a specificity of interest onto an immune effector cell or immunoresponsive cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a second generation CAR.

In certain non-limiting embodiments, the extracellular antigen-binding domain of the CAR (embodied, for example, an scFv or an analog thereof) binds to an antigen with a dissociation constant (K_{d}) of about 5×10⁻⁶ M or less. In certain embodiments, the K_{d} is about 5×10⁻⁶ M or less, about 1×10⁻⁶ M or less, 5×10⁻⁷ M or less, about 2×10⁻⁷ M or less, about 1×10⁻⁷ M or less, about 9×10⁻⁸M or less, about 1×10⁻⁸M or less, about 9×10⁻⁹M or less, about 5×10⁻⁹M or less, about 4×10⁻⁹M or less, about 3×10⁻⁹ or less, about 2×10⁻⁹M or less, or about 1×10⁻⁹M or less, or about 1×10⁻¹⁰ M or less. In certain non-limiting embodiments, the K_{d} is about 1×10⁻⁹M or less. In certain non-limiting embodiments, the K_{d} is about 1×10⁻¹⁰ M or less. In certain non-limiting embodiments, the K_{d} is from about 1×10⁻¹⁰ M to about 1×10⁻⁶ M. In certain non-limiting embodiments, the K_{d} is from about 1×10⁻⁹M to about 1×10⁻⁷ M. In certain non-limiting embodiments, the K_{d} is from about 1×10⁻¹⁰ M to about 1×10⁻⁷ M. In certain non-limiting embodiments, the K_{d} is from about 1×10⁻⁹M to about 1×10⁻⁷ M.

Binding of the extracellular antigen-binding domain (for example, in an scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

In accordance with the presently disclosed subject matter, a CARs can comprise an extracellular antigen-binding domain, a transmembrane domain and an intracellular signaling domain, wherein the extracellular antigen-binding domain specifically binds to an antigen, e.g., a tumor antigen or a pathogen antigen.

2.3.1. Extracellular Antigen Binding Domain of a CAR

In certain embodiments, the extracellular antigen-binding domain specifically binds to an antigen. In certain embodiments, the extracellular antigen-binding domain is an scFv. In certain embodiments, the scFv is a human scFv, a humanized scFv, or a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

In certain embodiments, the extracellular antigen-binding domain of a presently disclosed CAR is a murine scFv. In certain embodiments, the extracellular antigen-binding domain of a presently disclosed CAR is a murine scFv that binds to a murine CD19 polypeptide.

In certain embodiments, the extracellular antigen-binding domain of a presently disclosed CAR is an scFv that binds to a human CD19 polypeptide. In certain embodiments, the extracellular antigen-binding domain is a murine scFv, which comprises the amino acid sequence of SEQ ID NO: 10 and specifically binds to a human CD19 polypeptide. An exemplary nucleotide sequence encoding the amino acid sequence of SEQ ID NO: 10 is set forth in SEQ ID NO: 11. In certain embodiments, the murine scFv comprises a heavy chain variable region (V_{H}) comprising the amino acid sequence set forth in SEQ ID NO: 12. An exemplary nucleotide sequence encoding the amino acid sequence of SEQ ID NO: 12 is set forth in SEQ ID NO: 26. In certain embodiments, the murine scFV comprises a light chain variable region (V_{L}) comprising the amino acid sequence set forth in SEQ ID NO: 13. An exemplary nucleotide sequence encoding the amino acid sequence of SEQ ID NO: 13 is set forth in SEQ ID NO: 27. In certain embodiments, the murine scFV comprises V_{H} comprising the amino acid sequence set forth in SEQ ID NO: 12 and a V_{L} comprising the amino acid sequence set forth in SEQ ID NO: 13, optionally with (iii) a linker sequence, for example a linker peptide, between the V_{H} and the V_{L}.

In certain embodiments, the linker comprises amino acids having the sequence set forth in SEQ ID NO: 1. In certain embodiments, the extracellular antigen-binding domain comprises a V_{H} comprising an amino acid sequence that is at least about 80% (e.g., at least about 85%, at least about 90%, or at least about 95%) homologous or identical to SEQ ID NO: 12. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 12. In certain embodiments, the extracellular antigen-binding domain comprises a V_{H} comprising the amino sequence set forth in SEQ ID NO: 12. In certain embodiments, the extracellular antigen-binding domain comprises a V_{L} comprising an amino acid sequence that is at least about 80% (e.g., at least about 85%, at least about 90%, or at least about 95%) homologous or identical to the amino acid sequence set forth in SEQ ID NO: 13. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 13. In certain embodiments, the extracellular antigen-binding domain comprises a V_{L} comprising the amino acid sequence set forth in SEQ ID NO: 13. In certain embodiments, the extracellular antigen-binding domain comprises a V_{H} comprising an amino acid sequence that is at least about 80% (e.g., at least about 85%, at least about 90%, or at least about 95%) homologous or identical to the amino acid sequence set forth in SEQ ID NO: 12, and a V_{L} comprising an amino acid sequence that is at least about 80% (e.g., at least about 85%, at least about 90%, or at least about 95%) homologous or identical to the amino acid sequence set forth in SEQ ID NO: 13. In certain embodiments, the extracellular antigen-binding domain comprises a V_{H} comprising the amino acid sequence set forth in SEQ ID NO: 12 and a V_{L} comprising the amino acid sequence set forth in SEQ ID NO: 13.

In certain embodiments, the extracellular antigen-binding domain comprises a V_{H} CDR1 comprising the amino acid sequence set forth in SEQ ID NO: 14, or a conservative modification thereof, a V_{H} CDR2 comprising the amino acid sequence set forth in SEQ ID NO: 15 or a conservative modification thereof, and a V_{H} CDR3 comprising the amino acid sequence set forth in SEQ ID NO: 16, a conservative modification thereof. An exemplary nucleotide sequence encoding the amino acid sequence of SEQ ID NO: 14 is set forth in SEQ ID NO: 20. An exemplary nucleotide sequence encoding the amino acid sequence of SEQ ID NO: 15 is set forth in SEQ ID NO: 21. An exemplary nucleotide sequence encoding the amino acid sequence of SEQ ID NO: 16 is set forth in SEQ ID NO: 22.

In certain embodiments, the extracellular antigen-binding domain comprises a V_{H} CDR1 comprising the amino acid sequence set forth in SEQ ID NO: 14, a V_{H} CDR2 comprising the amino acid sequence set forth in SEQ ID NO: 15, and a V_{H} CDR3 comprising the amino acid sequence set forth in SEQ ID NO: 16.

In certain embodiments, the extracellular antigen-binding domain comprises a V_{L} CDR1 comprising the amino acid sequence set forth in SEQ ID NO: 17 or a conservative modification thereof, a V_{L} CDR2 comprising the amino acid sequence set forth in SEQ ID NO: 18 or a conservative modification thereof, and a V_{L} CDR3 comprising the amino acid sequence set forth in SEQ ID NO: 19 or a conservative modification thereof. An exemplary nucleotide sequence encoding the amino acid sequence of SEQ ID NO: 17 is set forth in SEQ ID NO: 23. An exemplary nucleotide sequence encoding the amino acid sequence of SEQ ID NO: 18 is set forth in SEQ ID NO: 24. An exemplary nucleotide sequence encoding the amino acid sequence of SEQ ID NO: 19 is set forth in SEQ ID NO: 25.

In certain embodiments, the extracellular antigen-binding domain comprises a V_{L} CDR1 comprising the amino acid sequence set forth in SEQ ID NO: 17, a V_{L} CDR2 comprising the amino acid sequence set forth in SEQ ID NO: 18, and a V_{L} CDR3 comprising the amino acid sequence set forth in SEQ ID NO: 19. In certain embodiments, the extracellular antigen-binding domain comprises a V_{H} CDR1 comprising the amino acid sequence set forth in SEQ ID NO: 14 or a conservative modification thereof, a V_{H} CDR2 comprising the amino acid sequence set forth in SEQ ID NO: 15 or a conservative modification thereof, a V_{H} CDR3 comprising the amino acid sequence set forth in SEQ ID NO: 16, a conservative modification thereof, a V_{L} CDR1 comprising the amino acid sequence set forth in SEQ ID NO: 17 or a conservative modification thereof, a V_{L} CDR2 comprising the amino acid sequence set forth in SEQ ID NO: 18 or a conservative modification thereof, and a V_{L} CDR3 comprising the amino acid sequence set forth in SEQ ID NO: 19 or a conservative modification thereof. In certain embodiments, the extracellular antigen-binding domain comprises a V_{H} CDR1 comprising amino acids having the sequence set forth in SEQ ID NO: 14, a V_{H} CDR2 comprising the amino acid sequence set forth in SEQ ID NO: 15, a V_{H} CDR3 comprising the amino acid sequence set forth in SEQ ID NO: 16, a V_{L} CDR1 comprising the amino acid sequence set forth in SEQ ID NO: 17, a V_{L} CDR2 comprising the amino acid sequence set forth in SEQ ID NO: 18 and a V_{L} CDR3 comprising the amino acid sequence set forth in SEQ ID NO: 19. SEQ ID NOS: 10-27 are provided in Table 1.

TABLE 1

| | | Mouse anti-human CD19 scFv | | |
|---|---|---|---|---|
| CDRs | | 1 | 2 | 3 |
| V_{H} | a.a. | GYAFSSY [SEQ ID | YPGDGD [SEQ ID NO: | KTISSVVDFYFDY [SEQ ID |
| | | NO: 14] | 15] | NO: 16] |
| | nt | Ggctatgcattcagtagc | Tatcctggagatggtgat | Aagaccattagttcggtagtag |
| | | tac [SEQ ID NO: | [SEQ ID NO: 21] | atttctactttgactac [SEQ |
| | | 20] | ID NO: 22] | |
| V_{L} | a.a. | KASQNVGTNVA [SEQ | SATYRNS [SEQ ID NO: | QQYNRYPYT [SEQ ID NO: |
| | | ID NO: 17] | 18] | 19] |
| | nt | Aaggccagtcagaatgtg | Tcggcaacctaccggaacagt | Caacaatataacaggtatccgt |
| | | ggtactaatgtagcc | [SEQ ID NO: 24] | acacg [SEQ ID NO: 25] |
| | | [SEQ ID NO: 23] | | |

| | | |
|---|---|---|
| Full | a.a. | EVKLQQSGAELVRPGSSVKISCKASGYAFSSYWMNWVKQRPGQGLEWIGQIYPGDGDTNYNGKFK |
| V_{H} | | GQATLTADKSSSTAYMQLSGLTSEDSAVYFCARKTISSVVDFYFDYWGQGTTVTVSS [SEQ ID |
| | | NO: 12] |
| | nt | Gaggtgaagctgcagcagtctggggctgagctggtgaggcctgggtcctcagtgaagatttcctg |
| | | caaggcttctggctatgcattcagtagctactggatgaactgggtgaagcagaggcctggacagg |
| | | gtcttgagtggattggacagatttatcctggagatggtgatactaactacaatggaaagttcaag |
| | | ggtcaagccacactgactgcagacaaatcctccagcacagcctacatgcagctcagcggcctaac |
| | | atctgaggactctgcggtctatttctgtgcaagaaagaccattagttcggtagtagatttctact |
| | | ttgactactggggccaagggaccacggtcaccgtctcctca [SEQ ID NO: 26] |
| Full | a.a. | DIELTQSPKFMSTSVGDRVSVTCKASQNVGTNVAWYQQKPGQSPKPLIYSATYRNSGVPDRFTGS |
| V_{L} | | GSGTDFTLTITNVQSKDLADYFCQQYNRYPYTSGGGTKLEIKR [SEQ ID NO: 13] |
| | nt | Gacattgagctcacccagtctccaaaattcatgtccacatcagtaggagacagggtcagcgtcac |
| | | ctgcaaggccagtcagaatgtgggtactaatgtagcctggtatcaacagaaaccaggacaatctc |
| | | ctaaaccactgatttactcggcaacctaccggaacagtggagtccctgatcgcttcacaggcagt |
| | | ggatctgggacagatttcactctcaccatcactaacgtgcagtctaaagacttggcagactattt |
| | | ctgtcaacaatataacaggtatccgtacacgtccggaggggggaccaagctggagatcaaacgg |
| | | [SEQ ID NO: 27] |
| scFv | a.a. | MALPVTALLLPLALLLHAEVKLQQSGAELVRPGSSVKISCKASGYAFSSYWMNWVKQRPGQGLEW |
| (including | | IGQIYPGDGDTNYNGKFKGQATLTADKSSSTAYMQLSGLTSEDSAVYFCARKTISSVVDFYFDYW |
| CD8a | | GQGTTVTVSSGGGGSGGGGSGGGGSDIELTQSPKFMSTSVGDRVSVTCKASQNVGTNVAWYQQKP |
| leader | | GQSPKPLIYSATYRNSGVPDRFTGSGSGTDFTLTITNVQSKDLADYFCQQYNRYPYTSGGGTKLE |
| sequence | | IKR [SEQ ID NO: 10] |
| | nt | Atggctctcccagtgactgccctactgcttcccctagcgcttctcctgcatgcagaggtgaagct |
| | | gcagcagtctggggctgagctggtgaggcctgggtcctcagtgaagatttcctgcaaggcttctg |
| | | gctatgcattcagtagctactggatgaactgggtgaagcagaggcctggacagggtcttgagtgg |
| | | attggacagatttatcctggagatggtgatactaactacaatggaaagttcaagggtcaagccac |
| | | actgactgcagacaaatcctccagcacagcctacatgcagctcagcggcctaacatctgaggact |
| | | ctgcggtctatttctgtgcaagaaagaccattagttcggtagtagatttctactttgactactgg |
| | | ggccaagggaccacggtcaccgtctcctcaggtggaggtggatcaggtggaggtggatctggtgg |
| | | aggtggatctgacattgagctcacccagtctccaaaattcatgtccacatcagtaggagacaggg |
| | | tcagcgtcacctgcaaggccagtcagaatgtgggtactaatgtagcctggtatcaacagaaacca |
| | | ggacaatctcctaaaccactgatttactcggcaacctaccggaacagtggagtccctgatcgctt |
| | | cacaggcagtggatctgggacagatttcactctcaccatcactaacgtgcagtctaaagacttgg |
| | | cagactatttctgtcaacaatataacaggtatccgtacacgtccggaggggggaccaagctggag |
| | | atcaaacgg [SEQ ID NO: 11] |

In certain embodiments, the extracellular antigen-binding domain of a presently disclosed CAR is an scFv that binds to a Sialyl Lewis A polypeptide. In certain embodiments, the scFv comprises a heavy chain variable region (V_{H}) comprising the amino acid sequence set forth in SEQ ID NO: 28. An exemplary nucleotide sequence encoding the amino acid sequence of SEQ ID NO: 28 is set forth in SEQ ID NO: 32. In certain embodiments, the scFv comprises a light chain variable region (V_{L}) comprising the amino acid sequence set forth in SEQ ID NO: 29. An exemplary nucleotide sequence encoding the amino acid sequence of SEQ ID NO: 29 is set forth in SEQ ID NO: 37. In certain embodiments, the scFv comprises V_{H} comprising the amino acid sequence set forth in SEQ ID NO: 28 and a V_{L} comprising the amino acid sequence set forth in SEQ ID NO: 29, optionally with (iii) a linker sequence, for example a linker peptide, between the V_{H} and the V_{L}. In certain embodiments, the linker comprises amino acids having the sequence set forth in SEQ ID NO: 1.

In certain embodiments, the extracellular antigen-binding domain comprises a V_{H} comprising an amino acid sequence that is at least about 80% (e.g., at least about 85%, at least about 90%, or at least about 95%) homologous or identical to the amino acid sequence set forth in SEQ ID NO: 28. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 28. In certain embodiments, the extracellular antigen-binding domain comprises a V_{H} comprising the amino acid sequence set forth in SEQ ID NO: 28. In certain embodiments, the extracellular antigen-binding domain comprises a V_{L} comprising an amino acid sequence that is at least about 80% (e.g., at least about 85%, at least about 90%, or at least about 95%) homologous or identical to the amino acid sequence set forth in SEQ ID NO: 29. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 29. In certain embodiments, the extracellular antigen-binding domain comprises a V_{L} comprising the amino acid sequence set forth in SEQ ID NO: 29. In certain embodiments, the extracellular antigen-binding domain comprises a V_{H} comprising an amino acid sequence that is at least about 80% (e.g., at least about 85%, at least about 90%, or at least about 95%) homologous or identical to the amino acid sequence set forth in SEQ ID NO: 28, and a V_{L} comprising an amino acid sequence that is at least about 80% (e.g., at least about 85%, at least about 90%, or at least about 95%) homologous or identical to the amino acid sequence set forth in SEQ ID NO: 29.

In certain embodiments, the extracellular antigen-binding domain comprises a V_{H} comprising the amino acid sequence set forth in SEQ ID NO: 28 and a V_{L} comprising the amino acid sequence set forth in SEQ ID NO: 29. In certain embodiments, the extracellular antigen-binding domain comprises a V_{H} CDR1 comprising the amino acid sequence set forth in SEQ ID NO: 30, or a conservative modification thereof, a V_{H} CDR2 comprising the amino acid sequence set forth in SEQ ID NO: 31 or a conservative modification thereof, and a V_{H} CDR3 comprising the amino acid sequence set forth in SEQ ID NO: 32, a conservative modification thereof. In certain embodiments, the extracellular antigen-binding domain comprises a V_{H} CDR1 comprising the amino acid sequence set forth in SEQ ID NO: 30, a V_{H} CDR2 comprising the amino acid sequence set forth in SEQ ID NO: 31, and a V_{H} CDR3 comprising the amino acid sequence set forth in SEQ ID NO: 32. In certain embodiments, the extracellular antigen-binding domain comprises a V_{L} CDR1 comprising the amino acid sequence set forth in SEQ ID NO: 33 or a conservative modification thereof, a V_{L} CDR2 comprising the amino acid sequence set forth in SEQ ID NO: 34 or a conservative modification thereof, and a V_{L} CDR3 comprising the amino acid sequence set forth in SEQ ID NO: 35 or a conservative modification thereof. In certain embodiments, the extracellular antigen-binding domain comprises a V_{L} CDR1 comprising the amino acid sequence set forth in SEQ ID NO: 33, a V_{L} CDR2 comprising the amino acid sequence set forth in SEQ ID NO: 34, and a V_{L} CDR3 comprising the amino acid sequence set forth in SEQ ID NO: 35. In certain embodiments, the extracellular antigen-binding domain comprises a V_{H} CDR1 comprising the amino acid sequence set forth in SEQ ID NO: 30 or a conservative modification thereof, a V_{H} CDR2 comprising the amino acid sequence set forth in SEQ ID NO: 31 or a conservative modification thereof, a V_{H} CDR3 comprising the amino acid sequence set forth in SEQ ID NO: 32, a conservative modification thereof, a V_{L} CDR1 comprising the amino acid sequence set forth in SEQ ID NO: 33 or a conservative modification thereof, a V_{L} CDR2 comprising the amino acid sequence set forth in SEQ ID NO: 34 or a conservative modification thereof, and a V_{L} CDR3 comprising the amino acid sequence set forth in SEQ ID NO: 35 or a conservative modification thereof. In certain embodiments, the extracellular antigen-binding domain comprises a V_{H} CDR1 comprising amino acids having the sequence set forth in SEQ ID NO: 30, a V_{H} CDR2 comprising the amino acid sequence set forth in SEQ ID NO: 31, a V_{H} CDR3 comprising the amino acid sequence set forth in SEQ ID NO: 32, a V_{L} CDR1 comprising the amino acid sequence set forth in SEQ ID NO: 33, a V_{L} CDR2 comprising the amino acid sequence set forth in SEQ ID NO: 34 and a V_{L} CDR3 comprising the amino acid sequence set forth in SEQ ID NO: 35. SEQ ID NOS: 28-37 are provided in Table 2.

TABLE 2

| Antigen | Sialyl Lewis A | | |
|---|---|---|---|
| CDRs | 1 | 2 | 3 |
| V_{H} | GFTFEAYA | INWNSGRI | AKDIRRFSTGGAEFEY [SEQ ID NO: 32] |
| | [SEQ ID NO: 30] | [SEQ ID NO: 31] | |
| V_{L} | SSNIGSNF | RNN | AAWDDSLGGHYV [SEQ ID NO: 35] |
| | [SEQ ID NO: 33] | [SEQ ID NO: 34] | |

| | |
|---|---|
| Full V_{H} | MEFGLSWLFL VAILKGVQCQ VQLVESGGGS VQPGRSLRLS CEASGFTFEA YAMHWVRQPP |
| | GKGLEWVSSI NWNSGRIAYA DSVKGRFTIS RDNARNSLYL QMNSLRLEDT AFYYCAKDIR |
| | RFSTGGAEFE YWGQGTLVTV SS [SEQ ID NO: 28] |
| DNA | ATGGAGTTTG GGCTGAGCTG GCTTTTTCTT GTGGCTATTT TAAAAGGCGT ACAGTGCCAG |
| | GTGCAGCTGG TGGAGTCTGG GGGAGGCTCG GTGCAGCCTG GCAGGTCCCT GAGACTCTCC |
| | TGTGAAGCCT CTGGATTCAC CTTTGAGGCC TATGCCATGC ACTGGGTCCG GCAACCTCCA |
| | GGGAAGGGCC TGGAGTGGGT CTCAAGTATT AATTGGAATA GTGGTCGCAT AGCCTATGCG |
| | GACTCTGTGA AGGGCCGATT CACCATCTCC AGAGACAACG CCAGGAATTC CCTGTATCTG |
| | CAAATGAACA GTCTGAGACT TGAGGACACG GCCTTCTATT ACTGTGCAAA AGATATACGG |
| | AGGTTTAGTA CCGGGGGGGC GGAGTTTGAG TACTGGGGCC AGGGAACCCT GGTCACCGTC |
| | TCCTCA [SEQ ID NO: 36] |
| Full V_{L} | MAGFPLLLTL LTHCAGSWAQ SVLTQPPSAS GTPGQRVTIS CSGSSSNIGS NFVYWYQQLP |
| | GTAPKLLIYR NNQRPSGVPD RFSGSRSGTS ASLAISGLRS EDEADYYCAA WDDSLGGHYV |
| | FGTGTKVTVL [SEQ ID NO: 29] |
| DNA | ATGGCCGGCT TCCCTCTCCT CCTCACCCTC CTCACTCACT GTGCAGGGTC TTGGGCCCAG |
| | TCTGTGCTGA CTCAGCCGCC CTCAGCGTCT GGGACCCCCG GGCAGAGGGT CACCATCTCT |
| | TGTTCTGGAA GCAGCTCCAA CATCGGAAGT AATTTTGTAT ACTGGTACCA GCAGCTCCCA |
| | GGAACGGCCC CCAAACTCCT CATATATAGG AATAATCAGC GGCCCTCAGG GGTCCCTGAC |
| | CGATTCTCTG GCTCCAGGTC TGGCACCTCA GCCTCCCTGG CCATCAGTGG ACTCCGGTCC |
| | GAGGATGAGG CTGATTATTA CTGTGCAGCA TGGGATGACA GCCTGGGAGG CCATTATGTC |
| | TTCGGAACTG GGACCAAGGT CACCGTCCTT [SEQ ID NO: 37] |

The V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 12, 13) may contain substitutions (e.g., conservative substitutions), insertions, or deletions relative to the specified sequence(s), but retain the ability to bind to a target antigen (e.g., CD19). In certain embodiments, a total of 1 to 10 amino acids are substituted, inserted and/or deleted in a specific sequence (e.g., SEQ ID NOs: 12 and 13). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from the group consisting of SEQ ID NOs: 12 and 13, including post-translational modifications of that sequence (SEQ ID NO: 12 and 13).

The V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 28 and 29) may contain substitutions (e.g., conservative substitutions), insertions, or deletions relative to the specified sequence(s), but retain the ability to bind to a target antigen (e.g., Sialyl Lewis A). In certain embodiments, a total of 1 to 10 amino acids are substituted, inserted and/or deleted in a specific sequence (e.g., SEQ ID NOs: 28 and 29). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from the group consisting of SEQ ID NOs: 28 and 29, including post-translational modifications of that sequence (SEQ ID NO: 28 and 29).

2.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a synthetic peptide (not based on a protein associated with the immune response), or a combination thereof.

In certain embodiments, the transmembrane domain comprises a CD8 polypeptide (e.g., a human CD8 polypeptide, e.g., a transmembrane domain of a human CD8 or a portion thereof). In certain embodiments, the CD8 polypeptide comprises or has an amino acid sequence that is at least about 85%, about 90%, about 95%, about 96%, about 97%, about 98%, about 99% or about 100% homologous or identical to the sequence having a NCBI Reference No: NP 001139345.1 (SEQ ID NO: 38) or a fragment thereof, and/or may optionally comprise up to one or up to two or up to three conservative amino acid substitutions. In certain embodiments, the CD8 polypeptide comprises or has an amino acid sequence that is a consecutive portion of SEQ ID NO: 38 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, or 200 to 235 of SEQ ID NO: 38. In certain embodiments, the CAR of the presently disclosed comprises a transmembrane domain comprising a CD8 polypeptide that comprises or has an amino acid sequence of amino acids 137 to 209 of SEQ ID NO: 38. SEQ ID NO: 38 is provided below.

[SEQ ID NO: 38]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the transmembrane domain comprises a murine CD8 polypeptide (e.g., a transmembrane domain of a murine CD8 or a portion thereof). In certain embodiments, the CD8 polypeptide comprises or has an amino acid sequence that is at least about 85%, about 90%, about 95%, about 96%, about 97%, about 98%, about 99% or about 100% homologous or identical to the sequence having a NCBI Reference No: AAA92533.1 (SEQ ID NO: 39) or a fragment thereof, and/or may optionally comprise up to one or up to two or up to three conservative amino acid substitutions. In certain embodiments, the CD8 polypeptide comprises or has an amino acid sequence that is a consecutive portion of SEQ ID NO: 39 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 39. In certain embodiments, the CAR of the presently disclosed comprises a transmembrane domain comprising a CD8 polypeptide that comprises or has an amino acid sequence of amino acids 151 to 219 of SEQ ID NO: 39. SEQ ID NO: 39 is provided below.

[SEQ ID NO: 39]

| | |
|---|---|
| 1 | MASPLTRFLS LNLLLMGESI ILGSGEAKPQ APELRIFPKK |
| | MDAELGQKVD LVCEVLGSVS |
| 61 | QGCSWLFQNS SSKLPQPTFV VYMASSHNKI TWDEKLNSSK |
| | LFSAVRDTNN KYVLTLNKFS |
| 121 | KENEGYYFCS VISNSVMYFS SVVPVLQKVN STTTKPVLRT |
| | PSPVHPTGTS QPQRPEDCRP |
| 181 | RGSVKGTGLD FACDIYIWAP LAGICVAPLL SLIITLICYH |
| | RSRKRVCKCP RPLVRQEGKP |
| 241 | RPSEKIV |

In certain embodiments, the CD8 polypeptide comprises or has the amino acid sequence set forth in SEQ ID NO: 40, which is provided below:

[SEQ ID NO: 40]
STTTKPVLRTPSPVHPTGTSQPQRPEDCRPRGSVKGTGLDFACDIYIWAP
LAGICVALLLSLIITLICY

In accordance with the presently disclosed subject matter, a “CD8 nucleic acid molecule” refers to a polynucleotide encoding a CD8 polypeptide.

An exemplary nucleotide sequence encoding the amino acid sequence of SEQ ID NO: 40 is set forth in SEQ ID NO: 41, which is provided below.

[SEQ ID NO: 41]
TCTACTACTACCAAGCCAGTGCTGCGAACTCCCTCACCTGTGCACCCTAC
CGGGACATCTCAGCCCCAGAGACCAGAAGATTGTCGGCCCCGTGGCTCAG
TGAAGGGGACCGGATTGGACTTCGCCTGTGATATTTACATCTGGGCACCC
TTGGCCGGAATCTGCGTGGCCCTTCTGCTGTCCTTGATCATCACTCTCAT
CTGCTAC

In certain embodiments, the transmembrane domain of a presently disclosed CAR comprises a CD28 polypeptide (e.g., a human CD28 polypeptide, e.g., a transmembrane domain of a human CD28). The CD28 polypeptide can have an amino acid sequence that is at least about 85%, about 90%, about 95%, about 96%, about 97%, about 98%, about 99% or 100% homologous or identical to the sequence having a NCBI Reference No: NP 006130 (SEQ ID NO: 42) or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 42 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, or 200 to 220 of SEQ ID NO: 42. In certain embodiments, the CD28 polypeptide comprised in the transmembrane domain of a presently disclosed CAR comprises or has an amino acid sequence of amino acids 153 to 179 of SEQ ID NO: 42.

SEQ ID NO: 42 is provided below:

[SEQ ID NO: 42]

| | |
|---|---|
| 1 | MLRLLLALNL FPSIQVTGNK ILVKQSPMLV AYDNAVNLSC |
| | KYSYNLFSRE FRASLHKGLD |
| 61 | SAVEVCVVYG NYSQQLQVYS KTGFNCDGKL GNESVTFYLQ |
| | NLYVNQTDIY FCKIEVMYPP |
| 121 | PYLDNEKSNG TIIHVKGKHL CPSPLFPGPS KPFWVLVVVG |
| | GVLACYSLLV TVAFIIFWVR |
| 181 | SKRSRLLHSD YMNMTPRRPG PTRKHYQPYA PPRDFAAYRS |

In certain embodiments, the transmembrane domain comprises a murine CD28 polypeptide (e.g., a transmembrane domain of a murine CD28 or a portion thereof). In certain embodiments, the CD28 polypeptide comprises or has an amino acid sequence that is at least about 85%, about 90%, about 95%, about 96%, about 97%, about 98%, about 99% or about 100% homologous or identical to the sequence having a NCBI Reference No: NP 031668.3 (SEQ ID NO: 43) or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 43 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 114 to 220, 150 to 200, 178 to 218, or 200 to 220 of SEQ ID NO: 43. In certain embodiments, the co-stimulatory signaling region of a presently disclosed CAR comprises a CD28 polypeptide that comprises or has the amino acids 151 to 177 of SEQ ID NO: 43.

SEQ ID NO: 43 is provided below:

[SEQ ID NO: 43]

| | |
|---|---|
| 1 | MTLRLLFLAL NFFSVQVTEN KILVKQSPLL VVDSNEVSLS |
| | CRYSYNLLAK EFRASLYKGV |
| 61 | NSDVEVCVGN GNFTYQPQFR SNAEFNCDGD FDNETVTFRL |
| | WNLHVNHTDI YFCKIEFMYP |
| 121 | PPYLDNERSN GTIIHIKEKH LCHTQSSPKL FWALVVVAGV |
| | LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide. An exemplary nucleotide sequence encoding amino acids 153 to 179 of SEQ ID NO: 42 is set forth in SEQ ID NO: 44, which is provided below.

[SEQ ID NO: 44]
TTTTGGGTGCTGGTGGTGGTTGGTGGAGTCCTGGCTTGCTATAGCTTGCT
AGTAACAGTGGCCTTTATTATTTTCTGGGTG

In certain embodiments, the transmembrane domain of the CAR comprises a transmembrane domain of human CD28 or a portion thereof. The transmembrane domain of human CD28 or a portion thereof can comprise or have an amino acid sequence that is at least about 85%, about 90%, about 95%, about 96%, about 97%, about 98%, about 99% or about 100% homologous or identical to the amnion acid sequence set forth in SEQ ID NO: 45, and/or may optionally comprise up to one or up to two or up to three conservative amino acid substitutions. SEQ ID NO: 45 is provided below:

[SEQ ID NO: 45]
FWVLVVVGGV LACYSLLVTV AFIIFWV.

An exemplary nucleic acid sequence encoding the amino acid sequence of SEQ ID NO: 45 is set forth in SEQ ID NO: 46, which is provided below.

[SEQ ID NO: 46]
TTTTGGGTGCTGGTGGTGGTTGGTGGAGTCCTGGCTTGCTATAGCTTGCT
AGTAACAGTGGCCTTTATTATTTTCTGGGTG

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3, a portion of a CD28 polypeptide (e.g., a portion of SEQ ID NO: 42), a portion of a CD8 polypeptide (e.g., a portion of SEQ ID NO: 38, or a portion of SEQ ID NO: 39), a variation of any of the foregoing which is at least about 80%, at least about 85%, at least about 90%, or at least about 95% homologous or identical thereto, or a synthetic spacer sequence.

2.3.3. Intracellular Signaling Domain of a CAR

In certain non-limiting embodiments, the intracellular signaling domain of the CAR comprises a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs. In certain embodiments, the CD3ζ polypeptide comprises or has an amino acid sequence that is at least about 85%, about 90%, about 95%, about 96%, about 97%, about 98%, about 99% or about 100% homologous or identical to the sequence having a NCBI Reference No: NP_932170 (SEQ ID NO: 47) or a fragment thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 47, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 100 to 150, or 150 to 164 of SEQ ID NO: 47. In certain embodiments, the CD3ζ polypeptide comprises or has an amino acid sequence of amino acids 52 to 164 of SEQ ID NO: 47.

SEQ ID NO: 47 is provided below:

[SEQ ID NO: 47]

| | |
|---|---|
| 1 | MKWKALFTAA ILQAQLPITE AQSFGLLDPK LCYLLDGILF |
| | IYGVILTALF LRVKFSRSAD |
| 61 | APAYQQGQNQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

In certain embodiments, the CD3ζ polypeptide comprises or has an amino acid sequence that is at least about 85%, about 90%, about 95%, about 96%, about 97%, about 98%, about 99% or about 100% homologous or identical to the sequence having a NCBI Reference No: NP_001106864.2 (SEQ ID NO: 48) or a fragment thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 48, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 52 to 142, 100 to 150, or 150 to 188 of SEQ ID NO: 48. In certain embodiments, the CD3ζ polypeptide comprises or has an amino acid sequence of amino acids 52 to 142 of SEQ ID NO: 48.

SEQ ID NO: 48 is provided below:

```
[SEQ ID NO: 48]
    1 MKWKVSVLAC ILHVRFPGAE AQSFGLLDPK LCYLLDGILF IYGVIITALY LRAKFSRSAE
  61 TAANLQDPNQ LYNELNLGRR EEYDVLEKKR ARDPEMGGKQ RRRNPQEGVY NALQKDKMAE
121 AYSEIGTKGE RRRGKGHDGL YQDSHFQAVQ FGNRREREGS ELTRTLGLRA RPKACRHKKP
181 LSLPAAVS
```

In certain embodiments, the CD3ζ polypeptide comprises or has the amino acid sequence set forth in SEQ ID NO: 49, which is provided below:

[SEQ ID NO: 49]
RAKFSRSAETAANLQDPNQLYNELNLGRREEYDVLEKKRARDPEMGGKQQR
RRNPQEGVYNALQKDKMAEAYSEIGTKGERRRGKGHDGLYQGLSTATKDTY
DALHMQTLAPR

In accordance with the presently disclosed subject matter, a “CD3ζ nucleic acid molecule” refers to a polynucleotide encoding a CD3ζ polypeptide. An exemplary nucleotide sequence encoding the amino acid sequence of SEQ ID NO: 49 is set forth in SEQ ID NO: 50, which is provided below.

[SEQ ID NO: 50]
AGAGCAAAATTCAGCAGGAGTGCAGAGACTGCTGCCAACCTGCAGGACCCC
AACCAGCTCTACAATGAGCTCAATCTAGGGCGAAGAGAGGAATATGACGTC
TTGGAGAAGAAGCGGGCTCGGGATCCAGAGATGGGAGGCAAACAGCAGAGG
AGGAGGAACCCCCAGGAAGGCGTATACAATGCACTGCAGAAAGACAAGATG
GCAGAAGCCTACAGTGAGATCGGCACAAAAGGCGAGAGGCGGAGAGGCAAG
GGGCACGATGGCCTTTACCAGGGTCTCAGCACTGCCACCAAGGACACCTAT
GATGCCCTGCATATGCAGACCCTGGCCCCTCGCTAA

In certain embodiments, the intracellular signaling domain of the CAR comprises a human CD3ζ polypeptide. The human CD3ζ polypeptide can comprise or have an amino acid sequence that is at least about 85%, about 90%, about 95%, about 96%, about 97%, about 98%, about 99% or about 100% homologous or identical to SEQ ID NO: 51 or a fragment thereof, and/or may optionally comprise up to one or up to two or up to three conservative amino acid substitutions. SEQ ID NO: 51 is provided below:

[SEQ ID NO: 51]
RVKFSRSADA PAYQQGQNQL YNELNLGRRE EYDVLDKRRG
RDPEMGGKPR RKNPQEGLYNELQKDKMAEA YSEIGMKGER
RRGKGHDGLY QGLSTATKDT YDALHMQALP PR.

An exemplary nucleic acid sequence encoding the amino acid sequence of SEQ ID NO: 51 is set forth in SEQ ID NO: 52, which is provided below.

[SEQ ID NO: 52]
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGG
CACGATGGCCTTTACCAGGGTCTCAGTACAGCCACCAAGGACACCTACGAC
GCCCTTCACATGCAGGCCCTGCCCCCTCGC

In certain non-limiting embodiments, the intracellular signaling domain of the CAR further comprises at least one co-stimulatory signaling region. In certain embodiments, the co-stimulatory region comprises at least one co-stimulatory molecule or a portion thereof (e.g., an intracellular domain of a co-stimulatory molecule or a portion thereof), which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide (e.g., an intracellular domain of CD28 or a portion thereof), a 4-1BB polypeptide (e.g., an intracellular domain of 4-1BB or a portion thereof), an OX40 polypeptide (e.g., an intracellular domain of OX40 or a portion thereof), an ICOS polypeptide (e.g., an intracellular domain of ICOS or a portion thereof), a DAP-10 polypeptide (e.g., an intracellular domain of DAP-10 or a portion thereof), or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide. In certain embodiments, the intracellular signaling domain of the CAR comprises a co-stimulatory signaling region that comprises a human CD28 polypeptide (e.g., an intracellular domain of human CD28 or a portion thereof). The CD28 polypeptide can comprise or have an amino acid sequence that is at least about 85%, about 90%, about 95%, about 96%, about 97%, about 98%, about 99% or 100% homologous or identical to the amino acid sequence set forth in SEQ ID NO: 42 or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 2 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, or 200 to 220 of SEQ ID NO: 42. In certain embodiments, the intracellular signaling domain of the CAR comprises a co-stimulatory signaling region that comprises a CD28 polypeptide comprising or having amino acids 180 to 220 of SEQ ID NO: 42.

In certain embodiments, the intracellular signaling domain of the CAR comprises a co-stimulatory signaling region that comprises a murine CD28 polypeptide (e.g., an intracellular domain of murine CD28 or a portion thereof). In certain embodiments, the CD28 polypeptide comprises or has an amino acid sequence that is at least about 85%, about 90%, about 95%, about 96%, about 97%, about 98%, about 99% or about 100% homologous or identical to the amino acid sequence SEQ ID NO: 43), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 43, which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 114 to 220, 150 to 200, 178 to 218, or 200 to 220 of SEQ ID NO: 43. In certain embodiments, the co-stimulatory signaling region of a presently disclosed CAR comprises a CD28 polypeptide that comprises or has the amino acids 178 to 218 of SEQ ID NO: 43.

In accordance with the presently disclosed subject matter, a “CD28 nucleic acid molecule” refers to a polynucleotide encoding a CD28 polypeptide. An exemplary nucleotide sequence that encodes amino acids 178 to 218 of SEQ ID NO: 43 is set forth in SEQ ID NO: 53, which is provided below.

[SEQ ID NO: 53]
AATAGTAGAAGGAACAGACTCCTTCAAAGTGACTACATGAACATGACTCCC
CGGAGGCCTGGGCTCACTCGAAAGCCTTACCAGCCCTACGCCCCTGCCAGA
GACTTTGCAGCGTACCGCCCC

In certain embodiments, the intracellular signaling domain of the CAR comprises an intracellular domain of human CD28. The intracellular signaling domain of human CD28 can comprise or have an amino acid sequence that is at least about 85%, about 90%, about 95%, about 96%, about 97%, about 98%, about 99% or about 100% homologous to SEQ ID NO: 54 or fragments thereof, and/or may optionally comprise up to one or up to two or up to three conservative amino acid substitutions. SEQ ID NO: 30 is provided below: RSKRSRLLHS DYMNMTPRRP GPTRKHYQPY APPRDFAAYR S [SEQ ID NO: 54].

An exemplary nucleic acid sequence encoding the amino acid sequence of SEQ ID NO: 54 is set forth in SEQ ID NO: 55, which is provided below.

[SEQ ID NO: 55]
AGGAGTAAGAGGAGCAGGCTCCTGCACAGTGACTACATGAACATGACTCCC
CGCCGCCCCGGGCCCACCCGCAAGCATTACCAGCCCTATGCCCCACCACGC
GACTTCGCAGCCTATCGCTCC

In certain embodiments, the intracellular signaling domain of the CAR comprises an intracellular domain of human CD28. The intracellular signaling domain of human CD28 can comprise or have an amino acid sequence that is at least about 85%, about 90%, about 95%, about 96%, about 97%, about 98%, about 99% or about 100% homologous to SEQ ID NO: 30 or fragments thereof, and/or may optionally comprise up to one or up to two or up to three conservative amino acid substitutions. SEQ ID NO: 56 is provided below:

[SEQ ID NO: 56]
AIEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPRD
FAAYRS.

An exemplary nucleic acid sequence encoding the amino acid sequence of SEQ ID NO: 56 is set forth in SEQ ID NO: 57, which is provided below.

[SEQ ID NO: 57]
GCAATTGAAGTTATGTATCCTCCTCCTTACCTAGACAATGAGAAGAGCAAT
GGAACCATTATCCATGTGAAAGGGAAACACCTTTGTCCAAGTCCCCTATTT
CCCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAGG
AGTAAGAGGAGCAGGCTCCTGCACAGTGACTACATGAACATGACTCCCCGC
CGCCCCGGGCCCACCCGCAAGCATTACCAGCCCTATGCCCCACCACGCGAC
TTCGCAGCCTATCGCTCC

In certain embodiments, the intracellular signaling domain of the CAR comprises a co-stimulatory signaling region that comprises two co-stimulatory molecules: CD28 and 4-1BB or CD28 and OX40.

In certain embodiments, the intracellular signaling domain of the CAR comprises a co-stimulatory signaling region that comprises a 4-1BB polypeptide (e.g., the intracellular domain of 4-1BB or a portion thereof). 4-1BB can act as a tumor necrosis factor (TNF) ligand and have stimulatory activity. The 4-1BB polypeptide can comprise or have an amino acid sequence that is at least about 85%, about 90%, about 95%, about 96%, about 97%, about 98%, about 99% or about 100% homologous or identical to the sequence having a NCBI Reference No: NP_001552 (SEQ ID NO: 58) or a fragment thereof, and/or may optionally comprise up to one or up to two or up to three conservative amino acid substitutions.

SEQ ID NO: 58 is provided below:

```
[SEQ ID NO: 58]
    1 MGNSCYNIVA TLLLVLNFER TRSLQDPCSN CPAGTFCDNN RNQICSPCPP NSFSSAGGQR
  61 TCDICRQCKG VFRTRKECSS TSNAECDCTP GFHCLGAGCS MCEQDCKQGQ ELTKKGCKDC
121 CFGTFNDQKR GICRPWTNCS LDGKSVLVNG TKERDVVCGP SPADLSPGAS SVTPPAPARE
181 PGHSPQIISF FLALTSTALL FLLFFLTLRF SVVKRGRKKL LYIFKQPFMR PVQTTQEEDG
241 CSCRFPEEEE GGCEL
```

In accordance with the presently disclosed subject matter, a “4-1BB nucleic acid molecule” refers to a polynucleotide encoding a 4-1BB polypeptide.

In certain embodiments, the intracellular signaling domain of the CAR comprises a co-stimulatory signaling region that an OX40 polypeptide (e.g., an intracellular domain of OX40 or a portion thereof). An OX40 polypeptide can comprise or have an amino acid sequence that is at least about 85%, about 90%, about 95%, about 96%, about 97%, about 98%, about 99% or about 100% homologous or identical to the sequence having a NCBI Reference No: NP_003318 (SEQ ID NO: 59), or fragments thereof, and/or may optionally comprise up to one or up to two or up to three conservative amino acid substitutions.

SEQ ID NO: 59 is provided below:

```
[SEQ ID NO: 59]
    1 MCVGARRLGR GPCAALLLLG LGLSTVTGLH CVGDTYPSND RCCHECRPGN GMVSRCSRSQ
  61 NTVCRPCGPG FYNDVVSSKP CKPCTWCNLR SGSERKQLCT ATQDTVCRCR AGTQPLDSYK
121 PGVDCAPCPP GHFSPGDNQA CKPWTNCTLA GKHTLQPASN SSDAICEDRD PPATQPQETQ
181 GPPARPITVQ PTEAWPRTSQ GPSTRPVEVP GGRAVAAILG LGLVLGLLGP LAILLALYLL
241 RRDQRLPPDA HKPPGGGSFR TPIQEEQADA HSTLAKI
```

In accordance with the presently disclosed subject matter, an “OX40 nucleic acid molecule” refers to a polynucleotide encoding an OX40 polypeptide.

In certain embodiments, the intracellular signaling domain of the CAR comprises a co-stimulatory signaling region that an ICOS polypeptide (e.g., an intracellular domain of ICOS or a portion thereof). An ICOS polypeptide can comprise or have an amino acid sequence that is at least about 85%, about 90%, about 95%, about 96%, about 97%, about 98%, about 99% or about 100% homologous or identical to the sequence having a NCBI Reference No: NP_036224 (SEQ ID NO: 60) or fragments thereof, and/or may optionally comprise up to one or up to two or up to three conservative amino acid substitutions.

SEQ ID NO: 60 is provided below:

```
[SEQ ID NO: 60]
    1 MKSGLWYFFL FCLRIKVLTG EINGSANYEM FIFHNGGVQI LCKYPDIVQQ FKMQLLKGGQ
  61 ILCDLIKTKG SGNTVSIKSL KFCHSQLSNN SVSFFLYNLD HSHANYYFCN LSIFDPPPFK
121 VTLIGGYLHI YESQLCCQLK FWLPIGCAAF VVVCILGCIL ICWLTKKKYS SSVHDPNGEY
181 MFMRAVNTAK KSRLTDVTL
```

In accordance with the presently disclosed subject matter, an “ICOS nucleic acid molecule” refers to a polynucleotide encoding an ICOS polypeptide.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

In certain embodiments, a presently disclosed CAR comprises an extracellular antigen-binding domain that binds to CD19 (e.g., human CD19), a transmembrane domain comprising a CD28 polypeptide (e.g., human CD28 polypeptide, e.g., a transmembrane domain of human CD28 or a portion thereof), and an intracellular signaling domain comprising a CD3ζ polypeptide (e.g., a human CD3ζ polypeptide) and a co-stimulatory signaling region. In certain embodiments, the CAR is a second generation CAR. In certain embodiments, the CAR is designated as “1928Z”. In certain embodiments, the CAR (e.g., 1928Z) comprises an amino acid sequence that is at least about 85%, about 90%, about 95%, about 96%, about 97%, about 98%, about 99% or about 100% homologous or identical to the amino acid sequence set forth in SEQ ID NO: 61, which is provided below. SEQ ID NO: 61 includes a CD8 leader sequence at amino acids 1 to 18, and is able to bind to CD19 (e.g., human CD19).

(SEQ ID NO: 61)
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAIEVMYPPPYLDNEKSNGTIIHVKGKHLCPSPLFP
GPSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR

An exemplary nucleic acid sequence encoding the amino acid sequence of SEQ ID NO: 61 is set forth in SEQ ID NO: 62, which is provided below.

(SEQ ID NO: 62)
ATGGCTCTCCCAGTGACTGCCCTACTGCTTCCCCTAGCGCTTCTCCTGCA
TGCAGAGGTGAAGCTGCAGCAGTCTGGGGCTGAGCTGGTGAGGCCTGGGT
CCTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTGG
ACAGATTTATCCTGGAGATGGTGATACTAACTACAATGGAAAGTTCAAGG
GTCAAGCCACACTGACTGCAGACAAATCCTCCAGCACAGCCTACATGCAG
CTCAGCGGCCTAACATCTGAGGACTCTGCGGTCTATTTCTGTGCAAGAAA
GACCATTAGTTCGGTAGTAGATTTCTACTTTGACTACTGGGGCCAAGGGA
CCACGGTCACCGTCTCCTCAGGTGGAGGTGGATCAGGTGGAGGTGGATCT
GGTGGAGGTGGATCTGACATTGAGCTCACCCAGTCTCCAAAATTCATGTC
CACATCAGTAGGAGACAGGGTCAGCGTCACCTGCAAGGCCAGTCAGAATG
TGGGTACTAATGTAGCCTGGTATCAACAGAAACCAGGACAATCTCCTAAA
CCACTGATTTACTCGGCAACCTACCGGAACAGTGGAGTCCCTGATCGCTT
CACAGGCAGTGGATCTGGGACAGATTTCACTCTCACCATCACTAACGTGC
AGTCTAAAGACTTGGCAGACTATTTCTGTCAACAATATAACAGGTATCCG
TACACGTCCGGAGGGGGGACCAAGCTGGAGATCAAACGGGCGGCCGCAAT
TGAAGTTATGTATCCTCCTCCTTACCTAGACAATGAGAAGAGCAATGGAA
CCATTATCCATGTGAAAGGGAAACACCTTTGTCCAAGTCCCCTATTTCCC
GGACCTTCTAAGCCCTTTTGGGTGCTGGTGGTGGTTGGTGGAGTCCTGGC
TTGCTATAGCTTGCTAGTAACAGTGGCCTTTATTATTTTCTGGGTGAGGA
GTAAGAGGAGCAGGCTCCTGCACAGTGACTACATGAACATGACTCCCCGC
CGCCCCGGGCCCACCCGCAAGCATTACCAGCCCTATGCCCCACCACGCGA
CTTCGCAGCCTATCGCTCCAGAGTGAAGTTCAGCAGGAGCGCAGACGCCC
CCGCGTACCAGCAGGGCCAGAACCAGCTCTATAACGAGCTCAATCTAGGA
CGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATG
AACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAG
TACAGCCACCAAGGACACCTACGACGCCCTTCACATGCAGGCCCTGCCCC
CTCGC

The presently disclosed subject matter also provides a nucleic acid composition comprising a first nucleic acid sequence encoding an antigen-recognizing receptor that binds to an antigen and a second nucleic acid sequence encoding an exogenous TRAIL polypeptide.

2.4. Truncated CAR

In certain embodiments, the antigen recognizing receptor is a truncated CAR. A “truncated CAR” is different from a CAR by lacking an intracellular signaling domain. Thus, a truncated CAR is a binding-but-non-signaling CAR. For example, a truncated CAR comprises an extracellular antigen-binding domain that binds to Sailyl Lewis A or CD19, and a transmembrane domain, and lacks an intracellular signaling domain (including a co-stimulatory signaling domain). In accordance with the presently disclosed subject matter, the truncated CAR can have a medium binding affinity to Sailyl Lewis A or CD19, in the K_{d} range of from about 1×10⁻⁹M to about 1×10⁻⁷ M. In accordance with the presently disclosed subject matter, the truncated CAR can have a high binding affinity to Sailyl Lewis A or CD19, in the K_{d} range of from about 1×10⁻¹⁰ M to about 1×10⁻⁸M. The truncated CAR functions as an adhesion molecule that enhances the avidity of a presently disclosed CAR and/or TCR, especially, one that has a low binding affinity to a hematological tumor-associated antigen, thereby improving the efficacy of the presently disclosed CAR and/or TCR or immunoresponsive cell (e.g., T cell) comprising thereof.

### 3. TNF-RELATED APOPTOSIS-INDUCING LIGAND (TRAIL)

The presently disclosed immunoresponsive cells comprises (a) an antigen-recognizing receptor disclosed herein (e.g., an antigen-recognizing receptor disclosed in Section 2), and (b) a secretable TRAIL polypeptide. In certain embodiments, the secretable TRAIL polypeptide is an exogenous TRAIL polypeptide.

TNF-related apoptosis-inducing ligand (TRAIL) (also known as TNF superfamily member 10, TL2, APO2L, CD253, Apo-2L, TNLG6A, GenBank ID: 8743 (human), 22035 (mouse), 246775 (rat)) is a gene encoding a cytokine that belongs to the tumor necrosis factor (TNF) ligand family, which can induce apoptosis in transformed and tumor cells. The protein product of TRAIL includes, but is not limited to, NCBI Reference Sequences NP_001177871.1, NP_001177872.1, and NP_003801.1.

In certain embodiments, a TRAIL polypeptide is to a bioactive form of TRAIL after secretion from a cell (e.g., a form where the signal peptide is cleaved off). In certain embodiments, the TRAIL polypeptide is a human TRAIL polypeptide. In certain embodiments, the human TRAIL polypeptide has the amino acid sequence set forth in SEQ ID NO: 63, which is provided below.

(SEQ ID NO: 63)
MAMMEVQGGP SLGQTCVLIV IFTVLLQSLC VAVTYVYFTN
ELKQMQDKYS KSGIACFLKE DDSYWDPNDE ESMNSPCWQV
WQLRQLVRK TPRMKRLWAA K

In certain embodiments, a secretable TRAIL polypeptide is a polypeptide or a protein, the cytokine portion of which has at least about 80%, about 85%, about 90%, about 95%, about 96%, about 97%, about 98%, about 99%, or about 100% homology or identity to the cytokine portion of the protein product of TRAIL (GenBank ID: 8743 (human), 22035 (mouse), 246775 (rat)), or a fragment thereof that has immunostimulatory activity. In certain non-limiting embodiments, the secretable TRAIL polypeptide comprises a cytokine portion and a signal peptide, optionally joined by a linker peptide. Non-limiting examples of secretable TRAIL polypeptides include NCBI Reference Sequences NP_001177871.1, NP_001177872.1, and NP_003801.1.

In certain non-limiting embodiments, the secretable TRAIL polypeptide comprises a signal peptide, for example, an IL-2 signal peptide, a kappa leader sequence, a CD8 leader sequence or a peptide with essentially equivalent activity. In certain embodiments, the secretable TRAIL polypeptide comprises an IL-2 signal peptide. In certain embodiments, the IL-2 signal peptide comprises or has the amino acid sequence set forth in SEQ ID NO: 2.

### 4. IMMUNORESPONSIVE CELLS

The presently disclosed immunoresponsive cells comprises (a) an antigen-recognizing receptor disclosed herein (e.g., an antigen-recognizing receptor disclosed in Section 2), and (b) a secretable TRAIL polypeptide. In certain embodiments, the antigen-recognizing receptor is capable of activating the immunoresponsive cell. In certain embodiments, the secretable TRAIL polypeptide (e.g., exogenous TRAIL polypeptide, such as a nucleic acid encoding a TRAIL polypeptide) is capable of promoting an anti-tumor effect of the immunoresponsive cell. The immunoresponsive cells can be transduced with an antigen-recognizing receptor and an exogenous TRAIL polypeptide such that the cells co-express the antigen-recognizing receptor and the exogenous TRAIL polypeptide.

In certain embodiments, the antigen-recognizing receptor (e.g., a CAR) targets the T-cell receptor a constant (TRAC) locus, and the expression of the antigen-recognizing receptor (e.g., a CAR) and the IL-1R1a is controlled by the native TCR alpha promoter elements, as disclosed in Eyquem J. et al Nature (2017); 543, 113-117, which is incorporated by reference in its entireties.

The presently disclosed subject matter further provides immunoresponsive cells comprising (a) an antigen-recognizing receptor (e.g., a CAR or a TCR) that binds to an antigen, and (b) a modified enhancer or promoter at an endogenous TRAIL gene. In certain embodiments, the modified enhancer or promoter enhances the gene expression of the endogenous TRAIL gene. In certain embodiments, the TRAIL coding sequence is provided in cis with the antigen-recognizing receptor (e.g., a CAR) in a bicistronic vector, and thus, both antigen-recognizing receptor (e.g., a CAR) and TRAIL are under the transcriptional control of one promoter (e.g., the retroviral SFG vector promoter). In certain embodiments, the endogenous TRAIL locus is modified to have induced transcription (e.g. by modifying the promoter or by providing/inducing upstream transcription factors that would result in the endogenous TRAIL gene expression).

The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and γδT cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the immunoresponsive cell is an NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, the immunoresponsive cells are cells of the myeloid lineage. Non-limiting examples of immunoresponsive cells of the myeloid lineage include macrophages, monocytes, neutrophils, basophils, eosinophils, erythrocytes, dendritic cells, and megakaryocytes or platelets. In certain embodiments, the immunoresponsive cell is macrophage.

The presently disclosed immunoresponsive cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including IL-10 and TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain non-limiting embodiments, the immunoresponsive cells can comprise and express (is transduced to express) a second antigen-recognizing receptor, which binds to a second antigen that is different than the antigen to which the first antigen-recognizing receptor binds. The second antigen can be a tumor antigen (e.g., any tumor antigens disclosed herein) or a pathogen antigen (e.g., any pathogen antigens disclosed herein).

### 5. COMPOSITIONS AND VECTORS

The presently disclosed subject matter provides compositions comprising an antigen-recognizing receptor disclosed herein (e.g., one disclosed in Section 2) and an exogenous TRAIL polypeptide (e.g., one disclosed in Section 3).

In certain embodiments, the exogenous TRAIL polypeptide is operably linked to a first promoter. In certain embodiments, the antigen-recognizing receptor is operably linked to a second promoter.

Furthermore, the presently disclosed subject matter provides nucleic acid compositions comprising a first polynucleotide encoding a TRAIL polypeptide disclosed herein (e.g., one disclosed in Section 3) and a second polynucleotide encoding an antigen-recognizing receptor disclosed herein (e.g., one disclosed in Section 2). Also provided are cells comprising such nucleic acid compositions.

In certain embodiments, the nucleic acid composition further comprises a first promoter that is operably linked to the TRAIL polypeptide. In certain embodiments, the nucleic acid composition further comprises a second promoter that is operably linked to the antigen-recognizing receptor.

In certain embodiments, one or both of the first and second promoters are endogenous or exogenous. In certain embodiments, the exogenous promoter is selected from the group consisting of an elongation factor (EF)-1 promoter, a CMV promoter, a SV40 promoter, a PGK promoter, a long terminal repeat (LTR) promoter and a metallothionein promoter. In certain embodiments, one or both of the first and second promoters are inducible promoters. In certain embodiments, the inducible promoter is selected from the group consisting of a NFAT transcriptional response element (TRE) promoter, a CD69 promoter, a CD25 promoter, an IL-2 promoter, an IL-12 promoter, a p40 promoter, and a Bcl-xL promoter.

The compositions and nucleic acid compositions can be administered to subjects or and/delivered into cells by art-known methods or as described herein. Genetic modification of an immunoresponsive cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of an immunoresponsive cell to include an antigen-recognizing receptors (e.g., CARs or TCRs), a retroviral vector is generally employed for transduction, however any other suitable viral vector or non-viral delivery system can be used. The antigen-recognizing receptor and the TRAIL polypeptide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify an immunoresponsive cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by microinjection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genomic DNA sequences.

Polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides). Immune cell may be targeted in vivo using recombinant tiviral or non-viral lymphotropiv paticles.

The resulting cells can be grown under conditions similar to those for unmodified cells, whereby the modified cells can be expanded and used for a variety of purposes.

### 6. ENHANCING ENDOGENOUS TRAIL GENE EXPRESSION

The presently disclosed subject matter provides immunoresponsive cells comprising an antigen-recognizing receptor disclosed herein (e.g., one disclosed in Section 2) and a modified enhancer or promoter at an endogenous TRAIL gene locus.

Any targeted genome editing methods (e.g., disclosed in Section 5) can be used to modify the promoter/enhancer region of a TRAIL gene locus, and thereby enhancing the endogenous expression of TRAIL in an immunoresponsive cell. In certain embodiments, the modification comprises replacement of an endogenous promoter with a constitutive promoter or an inducible promoter, or insertion of a constitutive promoter or inducible promoter to the promoter region of a TRAIL gene locus. In certain embodiments, a constitutive promoter is positioned on a TRAIL gene locus to drive gene expression of the endogenous TRAIL gene. Eligible constitutive promoters include, but are not limited to, a CMV promoter, an EF1a promoter, a SV40 promoter, a PGK1 promoter, a Ubc promoter, a beta-actin promoter, and a CAG promoter. Alternatively or additionally, a conditional or inducible promoter is positioned on a TRAIL gene locus to drive gene expression of the endogenous TRAIL gene. Non-limiting examples of conditional promoters include a tetracycline response element (TRE) promoter and an estrogen response element (ERE) promoter. In addition, enhancer elements can be placed in regions other than the promoter region.

Modification can be made anywhere within a TRAIL gene locus, or anywhere that can increase gene expression of a TRAIL gene. In certain embodiments, the modification occurs upstream of the transcriptional start site of a TRAIL gene. In certain embodiments, the modification occurs between the transcriptional start site and the protein coding region of a TRAIL gene. In certain embodiments, the modification occurs downstream of the protein coding region of a TRAIL gene. In certain embodiments, the modification occurs upstream of the transcriptional start site of a TRAIL gene, wherein the modification produces a new transcriptional start site.

### 7. POLYPEPTIDES AND ANALOGS

Also included in the presently disclosed subject matter are a CD19, CD28, 4-1BB, CD8, CD3, and TRAIL polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD19, CD28, 4-1BB, CD8, CD3, and TRAIL). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., .beta. or .gamma. amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., TRAIL). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 8. ADMINISTRATION

Compositions comprising the presently disclosed immunoresponsive cells or compositions comprising thereof can be provided systemically or directly to a subject for treating and/or preventing a neoplasia, a pathogen infection, or an infectious disease. In certain embodiments, the presently disclosed immunoresponsive cells or compositions comprising thereof are directly injected into an organ of interest (e.g., an organ affected by a neoplasia). Alternatively, the presently disclosed immunoresponsive cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed immunoresponsive cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed immunoresponsive cells can comprise a purified population of cells. Those skilled in the art can readily determine the percentage of the presently disclosed immunoresponsive cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed immunoresponsive cell), it can be formulated in a unit dosage injectable form (solution, suspension, emulsion).

### 9. FORMULATIONS

Compositions comprising the presently disclosed immunoresponsive cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In certain embodiments, between about 10⁴ and about 10¹⁰, between about 10⁵ and about 10⁹, or between about 10⁶ and about 10⁸, at least about 1×10⁵ of the presently disclosed immunoresponsive cells are administered to a subject. More effective cells may be administered in even smaller numbers. In certain embodiments, at least about 1×10⁵, at least about 2×10⁵, at least about 5×10⁵, at least about 1×10⁶, at least about 1×10⁷, at least about 1×10⁸, about 2×10⁸, about 3×10⁸, about 4×10⁸, or about 5×10⁸ of the presently disclosed immunoresponsive cells are administered to a subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 10. METHODS OF TREATMENTS

The presently disclosed immunoresponsive cells and compositions comprising thereof can be used for treating and/or preventing a neoplasia in a subject. The presently disclosed immunoresponsive cells and compositions comprising thereof can be used for prolonging the survival of a subject suffering from a neoplasia. The presently disclosed immunoresponsive cells and compositions comprising thereof can also be used for treating and/or preventing a pathogen infection or other infectious disease in a subject, such as an immunocompromised human subject. Such methods comprise administering the presently disclosed immunoresponsive cells in an amount effective or a composition (e.g., a pharmaceutical composition) comprising such immunoresponsive cells to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

The presently disclosed subject matter further provides methods of reducing tumor burden or treating and/or preventing neoplasia in a subject. In certain embodiments, the method comprises: i) administering to the subject a radiation therapy; and ii) administering to the subject an effective amount of immunoresponsive cells or a pharmaceutical composition comprising thereof.

In certain embodiments, the radiation therapy is a low dose radiation therapy. In certain embodiments, the radiation therapy is administered in a total dose of no more than or up to about 100 Gy, about 80 Gy, about 60 Gy, about 40 Gy, about 30 Gy, about 25 Gy, about 20 Gy, about 19 Gy, about 18 Gy, about 17 Gy, about 16 Gy, about 15 Gy, about 14 Gy, about 13 Gy, about 12 Gy, about 11 Gy, about 10 Gy, about 9 Gy, about 8 Gy, about 7 Gy, about 6 Gy, about 5 Gy, about 4 Gy, about 3 Gy, about 2.5 Gy, about 2 Gy, about 1.5 Gy, about 1 Gy or about 0.5 Gy. In certain embodiments, the radiation therapy is administered in a total dose of between about 0.01 Gy and about 100 Gy, between about 0.1 Gy and about 50 Gy, between about 0.1 Gy and about 30 Gy, between about 0.5 Gy and about 30 Gy, between about 1 Gy and about 50 Gy, between about 1 Gy and about 40 Gy, between about 1 Gy and about 30 Gy, between about 2 Gy and about 50 Gy, between about 2 Gy and about 40 Gy, between about 2 Gy and about 30 Gy, between about 2 Gy and about 20 Gy, between about 1 Gy and about 20 Gy, between about 2 Gy and about 15 Gy, between about 5 Gy and about 20 Gy, between about 5 Gy and about 15 Gy, between about 10 Gy and about 50 Gy, or between about 10 Gy and about 30 Gy. In certain embodiments, the radiation therapy is administered in a total dose of between about 1 Gy and about 20 Gy. In certain embodiments, the total dose is administered in about 1 fraction, about 2 fractions, about 3 fractions, about 4 fractions, about 5 fractions, about 6 fractions, about 7 fractions, about 8 fractions, about 9 fractions, about 10 fractions, about 11 fractions, about 12 fractions, about 13 fractions, about 14 fractions, about 15 fractions or more.

In certain embodiments, the radiation therapy is administered no later than about 50 days, about 40 days, about 30 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, about 1 day or about 0.5 day prior to the administration of the immunoresponsive cells or composition. In certain embodiments, the radiation therapy is administered no later than about 10 days prior to the administration of the immunoresponsive cells. In certain embodiments, the radiation therapy is administered between about 0.5 day and about 20 days prior to the administration of the immunoresponsive cells or composition, between about 1 day and about 20 days prior to the administration of the immunoresponsive cells or composition, between about 1 day and about 15 days prior to the administration of the immunoresponsive cells or composition, between about 2 day and about 20 days prior to the administration of the immunoresponsive cells or composition, between about 3 day and about 20 days prior to the administration of the immunoresponsive cells or composition, between about 2 day to about 15 days prior and the administration of the immunoresponsive cells or composition, or between about 2 day to about 10 days prior to the administration of the immunoresponsive cells or composition.

In certain embodiments, the radiation therapy is an external beam radiation therapy, a brachytherapy or a systemic radioisotope therapy. In certain embodiments, the immunoresponsive cell comprises an antigen recognizing receptor. In certain embodiments, the immunoresponsive cell is any immunoresponsive cell disclosed herein.

For adoptive immunotherapy using antigen-specific T cells, cell doses in the range of about 10⁵-10¹⁰ (e.g., at least about 1×10⁵, at least about 1×10⁶, e.g., about 10⁹) are typically infused. Upon administration of the presently disclosed cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal and directly to the thymus.

The presently disclosed subject matter provides methods for treating and/or preventing a neoplasia in a subject. The method can comprise administering an effective amount of the presently disclosed immunoresponsive cells or a composition comprising thereof to a subject having a neoplasia.

Non-limiting examples of neoplasia include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed immunoresponsive cells and compositions comprising thereof can be used for treating and/or preventing blood cancers (e.g., leukemias, lymphomas, and myelomas) or ovarian cancer, which are not amenable to conventional therapeutic interventions.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the immunoresponsive cells described herein in treatment prophylactically to prevent the occurrence of neoplasia until it is suitable to perform preventive surgery.

As a consequence of the surface expression of an antigen-recognizing receptor that binds to a tumor antigen and a secretable TRAIL polypeptide (e.g., an exogenous TRAIL polypeptide), adoptively transferred immunoresponsive cells (e.g., T cells) are endowed with enhanced tumor killing effects. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK−, NKT− cells, dendritic cells, and macrophages).

The presently disclosed subject matter further provides methods of reducing tumor burden, and/or treating and/or preventing a neoplasia in a subject, the method comprising: i) administering to the subject a radiation therapy; ii) administering to the subject an effective amount of an immunoresponsive cells comprising an antigen-recognizing receptor (e.g., a CAR); and iii) administering to the subject an effective amount of a TRAIL mimetic. In certain embodiments, the TRAIL mimetic is selected from the group consisting of a natural ligand of TRAIL receptor (e.g., TRAIL), a TRAIL analog, a TRAIL-specific antibody or an antigen-binding fragment thereof (e.g., a scFv) or VHH. In certain embodiments, the radiation therapy is administered prior to the administration of the immunoresponsive cells. In certain embodiments, the antigen-recognizing receptor is a CAR. In certain embodiments, the antigen-recognizing receptor is one disclosed in Section 2.

In certain embodiments, the TRAIL mimetic is administered concurrently, before, or after the administration of the immunoresponsive cells.

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed immunoresponsive cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

### 11. KITS

The presently disclosed subject matter provides kits for treating and/or preventing a neoplasia or a pathogen infection in a subject. In certain embodiments, the kit comprises an effective amount of the presently disclosed immunoresponsive cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding an TRAIL polypeptide in expressible (and secretable) form, which may optionally be comprised in the same or different vectors.

If desired, the immunoresponsive cells and/or nucleic acid molecules are provided together with instructions for administering the cells or nucleic acid molecules to a subject having or at risk of developing a neoplasia or pathogen or immune disorder. The instructions generally include information about the use of the composition for the treatment and/or prevention of neoplasia or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

Introduction

Strategies that improve antigen presentation, induce epitope spreading, or perpetuate existing antitumor T cell responses hold promise for combating tumor antigen escape. For example, cancer vaccines and “immunogenic” radiation (RT) activate antigen-presenting cells (APCs) to improve tumor neoantigen display to endogenous T cells. However, the same neoantigens must still be expressed and presented in most, if not all, tumor cells in order to obtain a complete response. In patients who have pre-existing tumor-reactive T cells, which correlates with tumor mutational burden, immune checkpoint inhibitors can relieve T cell exhaustion and provide sustained responses. However, checkpoint inhibition cannot restore T cell responses against tumor cells that do not present the recognized antigens, just as CARs cannot direct a response against tumor cells devoid of the CAR target.

The improved tumor recognition that can occur after exposure to ionizing radiation, mediated by increased APC activation, improved T cell infiltration, and enhanced HLA or CAR target expression on the tumor (Spiotto et al., Sci Immunol (2016); 1; Weiss et al., Cancer Res (2018); 78:1031-1043), faces the same challenge of antigen escape owing to antigen loss. However, it was found that tumors that have been exposed to low dose irradiation become more sensitive to CAR T cell activity, including tumor cells that lack the CAR target. Understanding this mechanism may be particularly valuable in overcoming solid tumor antigen escape.

This alternative mechanism was characterized by which tumor susceptibility to CAR T cell-mediated elimination is enhanced by radiation conditioning, and exploit it to extend the reach of CAR T cells beyond the targeted antigen. Pancreatic cancer continues to carry a dismal

prognosis with little improvement over the last decades, does not have established uniformly expressed therapeutic target antigens, and is increasing in incidence. In an orthotopic pancreatic cancer model that is partially antigen-negative, a novel means was provided to address the challenge of clonal antigen heterogeneity by combining low-dose radiation and CAR therapy.

Results

Low Dose RT Sensitizes Nalm6 ALL to CAR T Cell Killing, In Vitro and In Vivo

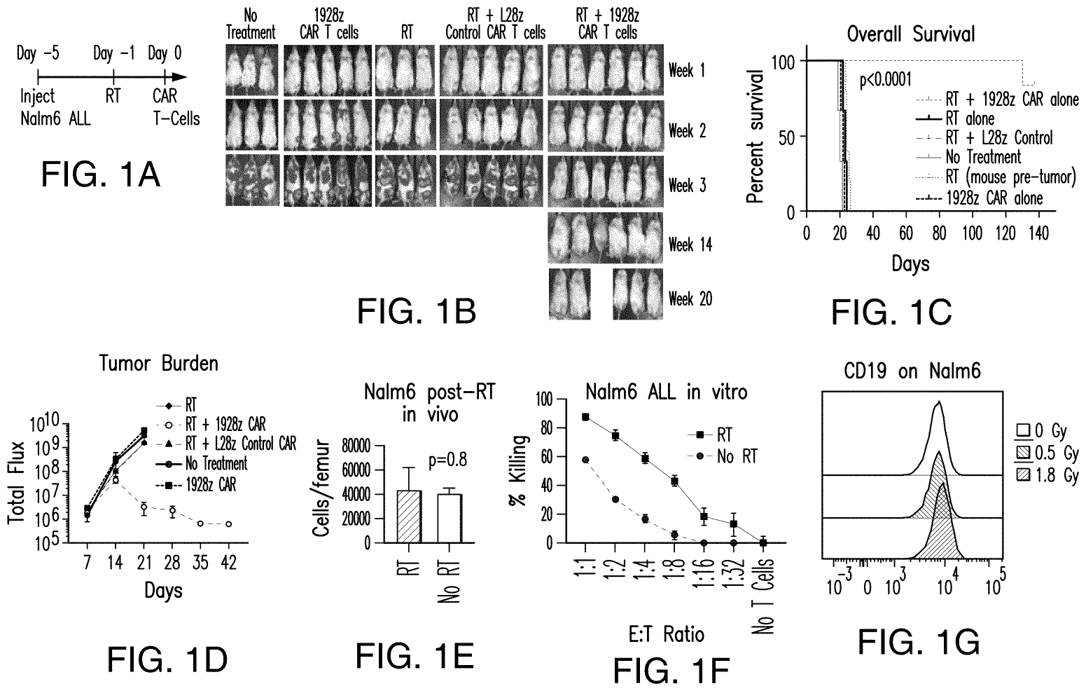

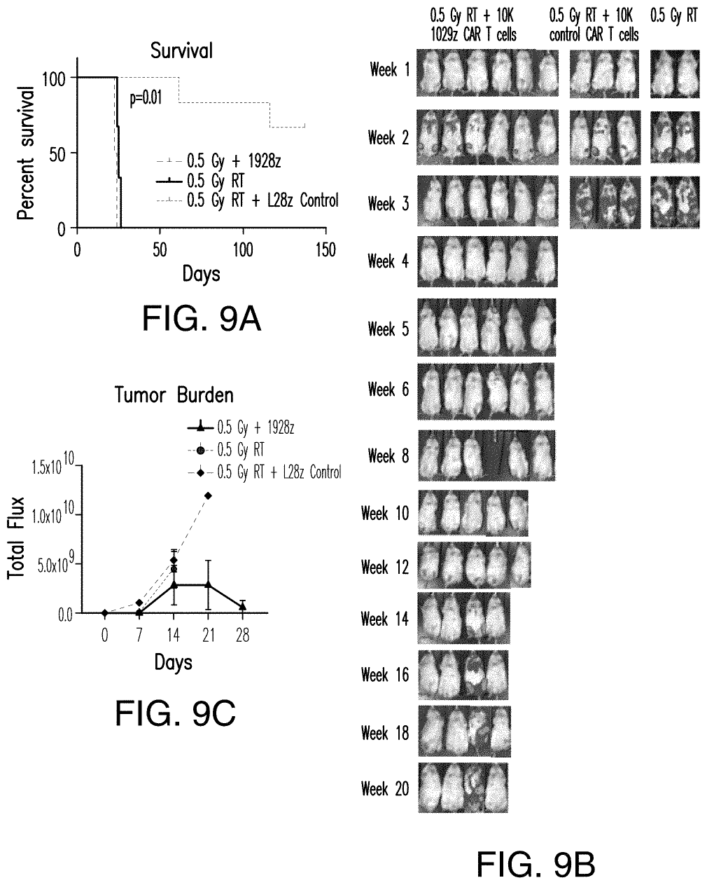

The effect of RT in the well-established CD19⁺ NALM6 leukemia model (Zhao et al., Cancer Cell (2015); 28:415-428) was first evaluated by assessing whether RT conditioning affects the therapeutic efficacy of 1928z CAR T cells in leukemia-bearing mice. Mice harboring NALM6 were administered 10⁴ 1928 z CAR T cells, a suboptimal T cell dose (Zhao et al., Cancer Cell (2015); 28:415-428) and/or 1.8 Gy, a small but clinically relevant radiation dose hereafter referred to as “low-dose RT” (FIG. 1A). Whereas mice given either treatment alone succumbed to disease within 3 weeks, those given CART cells after RT survived >100 days (FIGS. 1B-1D). A lower dose of 0.5 Gy to the tumor-bearing animals before CAR T cells still conferred a significant survival advantage (FIGS. 9A-9C). Quantification of tumor cells showed a similar tumor burden whether mice were exposed to low-dose RT or not, suggesting the effect is not dependent on tumor debulking (FIG. 1E). Irradiating mice before infusing tumor and CAR T cells provided no benefit (FIG. 1C), suggesting that the tumor had to be exposed to RT. Indeed, in vitro cytotoxicity assays performed on viable RT-exposed or RT-naïve tumor cells revealed greater killing of RT-exposed tumor, despite identical levels of target antigen (FIGS. 1F-1G).

Low Dose RT Induces Surface Expression of Death Receptor 5

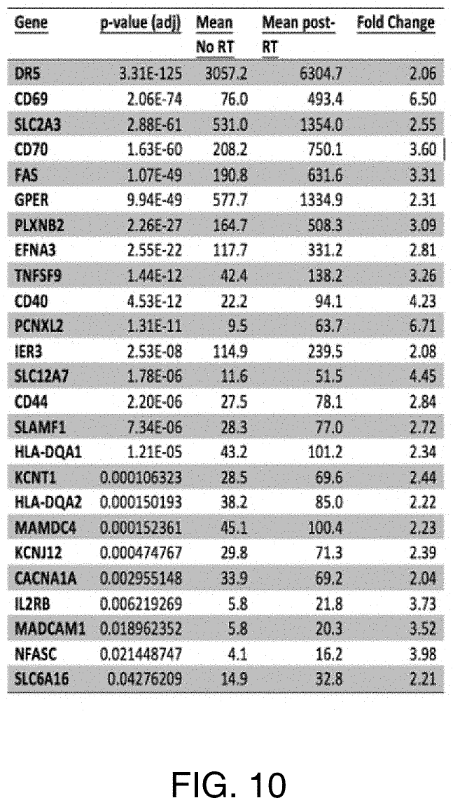

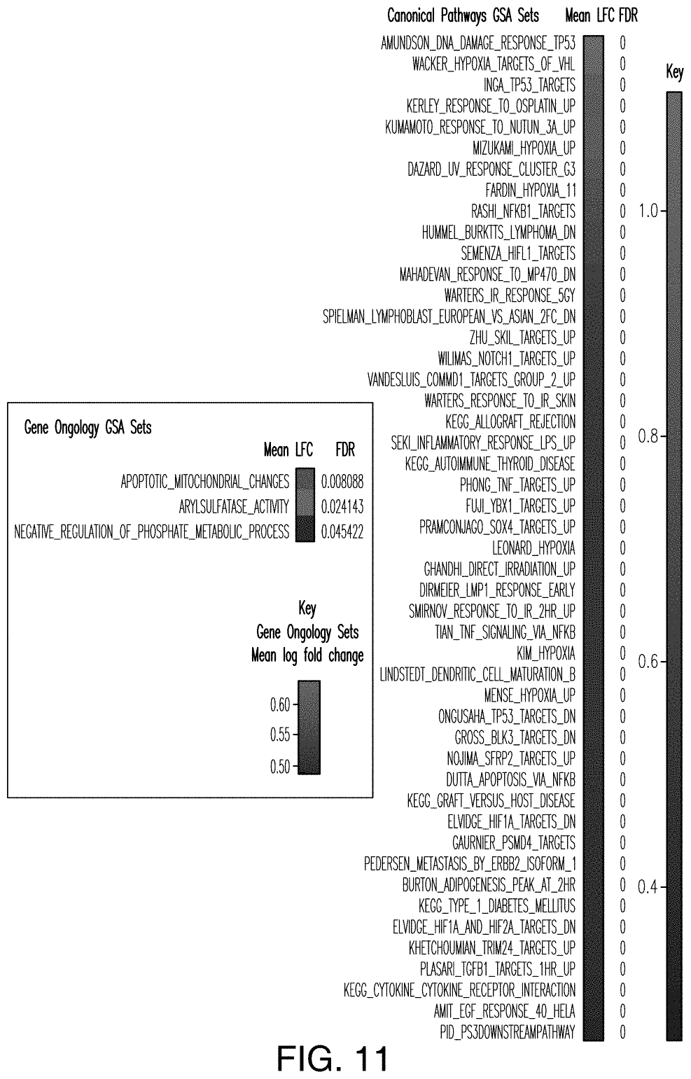

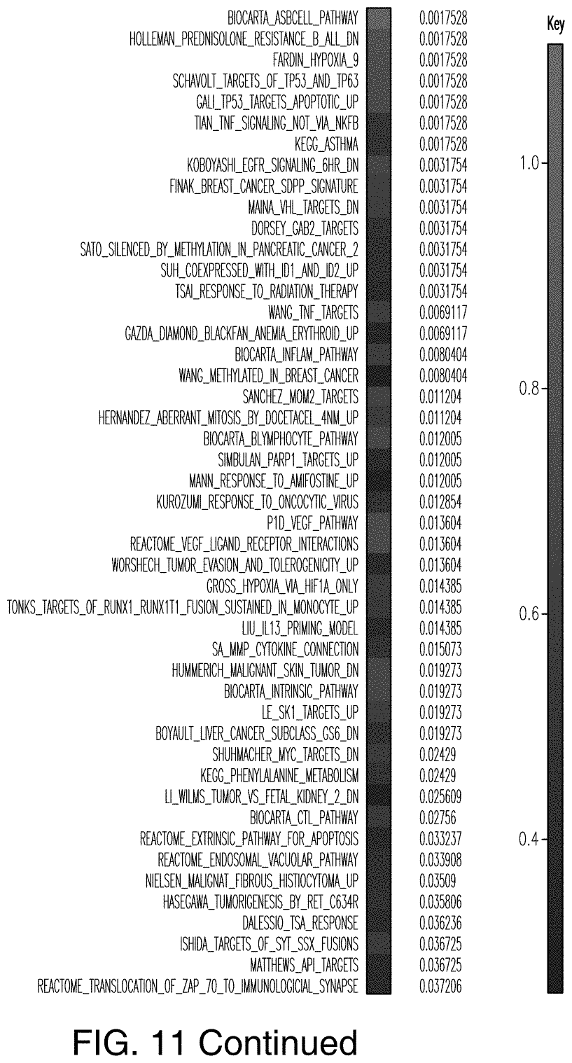

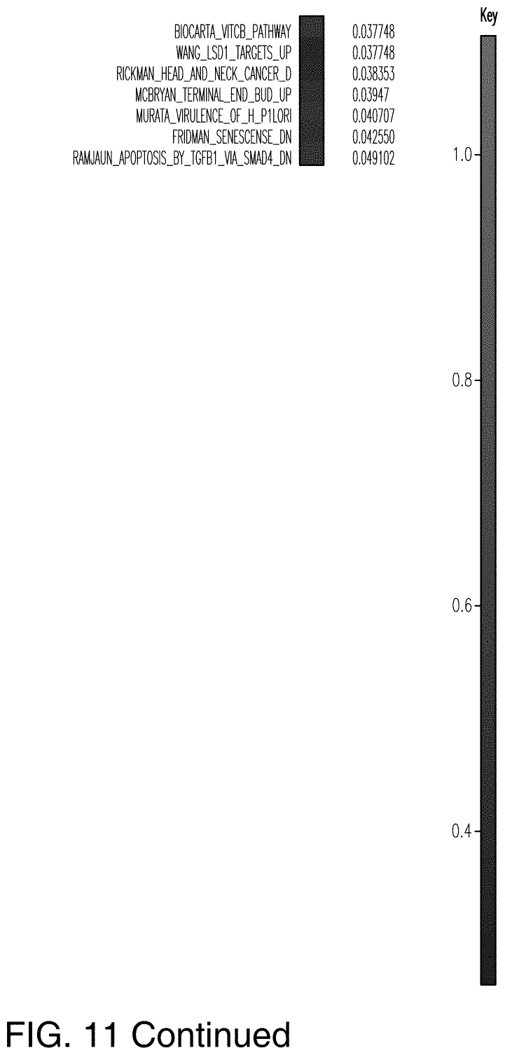

To gain insights into pathways and molecules mediating CAR T cell sensitization, RNAseq was performed on NALM6 tumor cells 6 hours after exposure to low-dose subtherapeutic RT of 0.5 Gy. 123 individual genes were induced two-fold or higher, 24 of which were identified as surface proteins (FIG. 10). The most statistically significant membrane molecule induced was Death Receptor 5 (DR5; TRAIL R2) (p=10⁻¹²⁵) (FIG. 10). Gene set analysis (GSA) did not reveal significant changes in any Immunologic Signatures. However, there were significant increases in pro-apoptotic gene sets such as the Gene Ontology Apoptotic Mitochondrial Changes (adjusted p=0.008) (FIG. 10), and the canonical pathway Extrinsic Pathway for Apoptosis (adjusted p=0.03) (FIG. 10). Both of these pathways are utilized by death receptors.

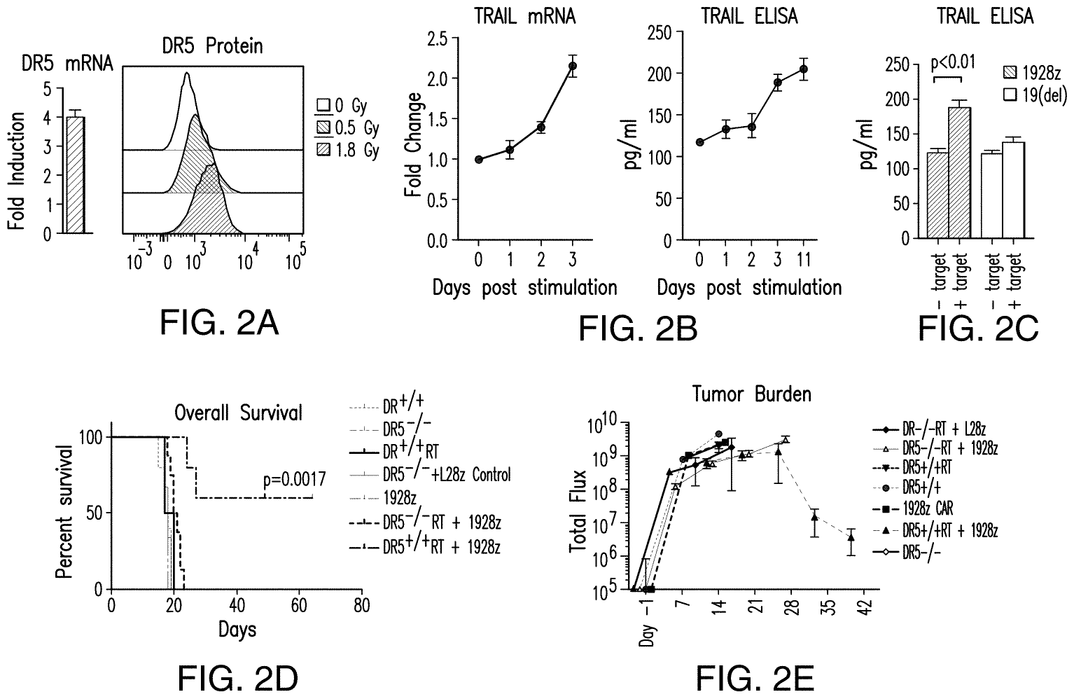

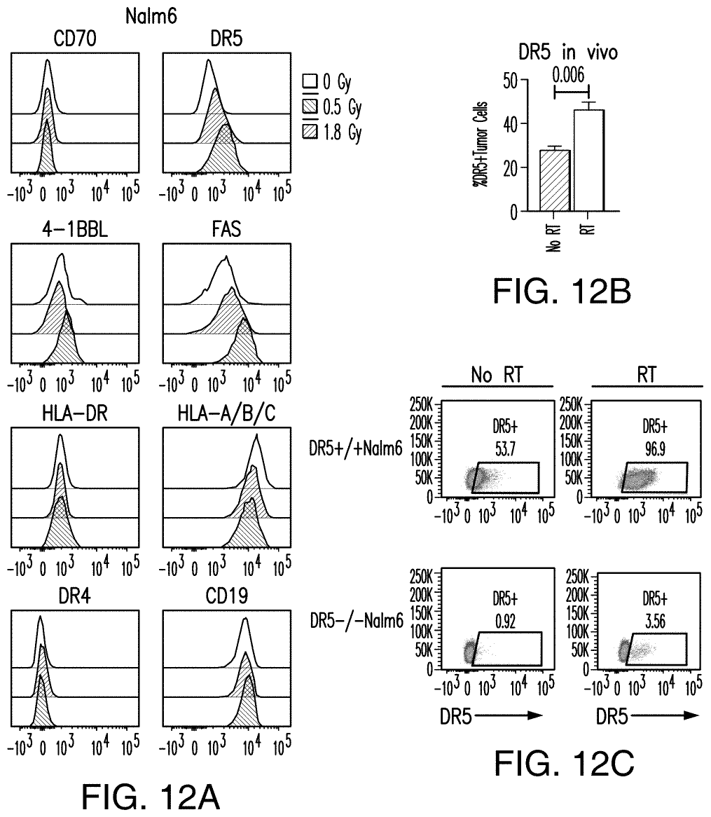

Surface expression of proteins with potential to interact with molecules produced by T cells was assessed by FACS analysis (FIGS. 12A-12C). Some potentially facilitative proteins, such as 4-1BBL, were expressed at very low baseline RNA levels and thus despite increases in RNA detection were not significantly increased at the protein level. DR5, but not related Death Receptor 4, surface protein significantly increased after low-dose RT (FIG. 2A and FIG. 12A). DR5 was induced both in vitro and in vivo following low dose RT (FIG. 12B).

CAR T Cells Express TRAIL, the Ligand for DR5, Upon Antigen Encounter

It was hypothesized that sensitization to CAR T cell killing may occur through induction of a tumor cell protein or pathway that interacts with a cognate ligand or receptor on the CAR T cell. Since DR5 and its downstream signaling cascade are transcriptionally induced, expression of TRAIL, the ligand for DR5 by CAR T cells, was examined. CAR T cells produce low levels of TRAIL at baseline, however upon target antigen encounter, significantly induce TRAIL mRNA and protein (FIG. 2B). In contrast, TRAIL is not induced after tumor recognition by T cells expressing a truncated CAR that lacks the signaling domain (Ldel), establishing the dependence of TRAIL induction on CAR signaling (FIG. 2C).

DR5 is an Important Mediator of RT Sensitization to CAR T Cell Killing

Given that TRAIL can induce apoptosis through DR5 (Wang et al., Oncogene (2003); 22:8628-8633) in a concentration dependent manner (Hartwig et al., Mol Cell (2017); 65:730-742 e735), whether DR5 pathway induction after low dose RT may functionally sensitize tumor cells to CAR T cell-mediated killing was tested. Since all of the DR5 signaling molecules are shared with other apoptotic pathways except the receptor itself, DR5 expression on NALM6 was altered. Overexpressing DR5 followed by sorting the DR5-high population only resulted in temporary up-regulation as DR5-high cells reverted to baseline endogenous levels after three weeks in culture. However, stable DR5^{-/-} cells was generated using CRISPR gene editing (FIG. 12C). DR5^{wt} and DR5^{-/-} tumor bearing animals succumb to disease within three weeks, which is unaffected by low dose CAR T cells or RT alone. Importantly, the survival benefit observed with sensitizing RT+CAR T cells against DR5′ tumor was no longer achieved in DR5 tumor bearing mice (FIGS. 2D-2E).

RT and CAR T Cell Timing of Delivery is Important In Vivo

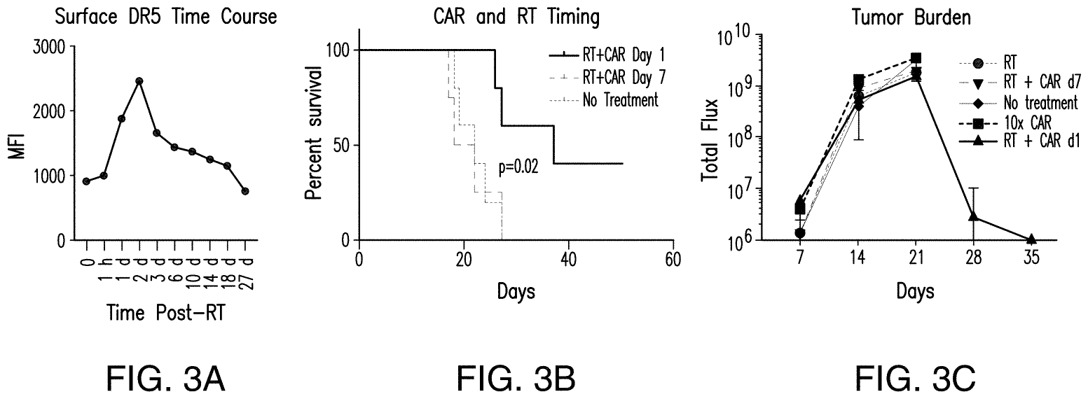

To establish the time window for administering CAR T cells after RT, DR5 levels on NALM6 cells were tested after low dose RT over time, and found peak expression after two days, which returned to baseline levels by one month (FIG. 3A). In vivo, mice treated with CAR T cells one day after sensitizing RT had significant survival advantage over those treated one week after RT (FIGS. 3B-3C), suggesting that early administration of CAR T cell therapy after RT is optimal.

Generation of a Novel CAR Against Pancreatic Cancer

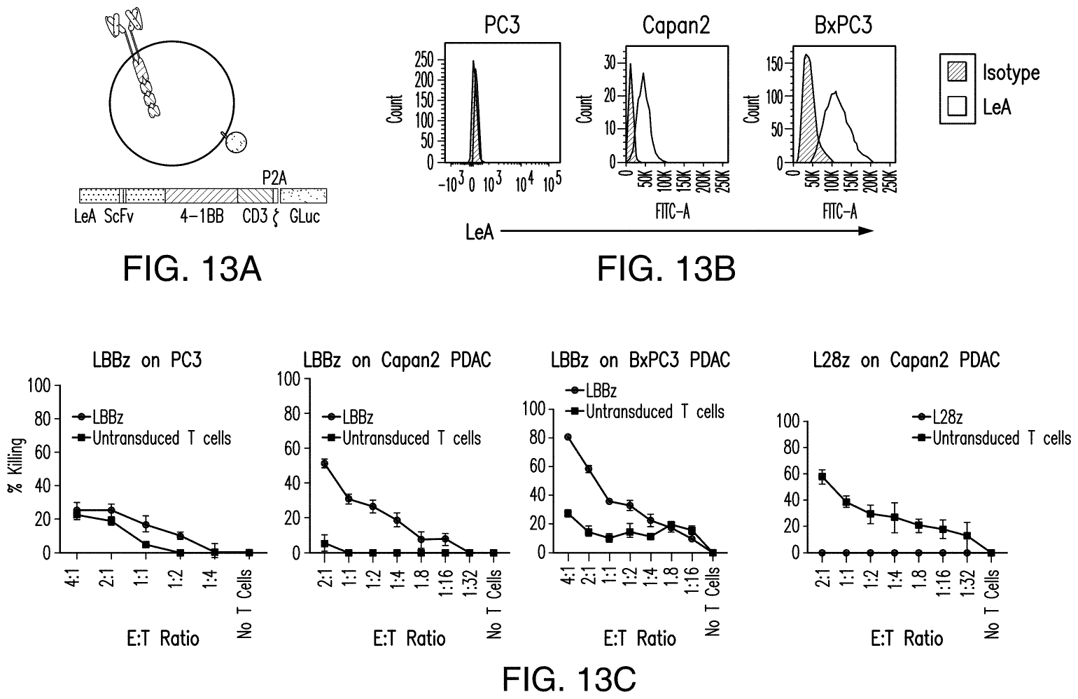

Sensitizing solid tumors to CAR T cells may help extend the potential benefit of CAR therapy to a wider range of cancer patients. Pancreatic ductal adenocarcinoma (PDAC) is particularly resilient against both conventional and immune therapies, and continues to carry a poor prognosis at all stages of diagnosis. While it is difficult to identify a target that is consistently expressed on PDAC and not on critical normal tissues, sialyl Lewis A (Le^{A}) has been recognized as an attractive antibody target as it is a surface antigen expressed on 75-90% of pancreatic tumors (Viola-Villegas et al., J Nucl Med (2013); 54:1876-1882) with low expression on normal human tissues (Viola-Villegas et al., J Nucl Med (2013); 54:1876-1882). Three PDAC clinical trials are currently open targeting Le^{A} with radiolabeled antibody (NCT03118349, NCT02672917, NCT02687230). The human monoclonal 5B1 antibody targeting Le^{A} has demonstrated specificity for pancreatic cancer in vitro and in vivo (Viola-Villegas et al., J Nucl Med (2013); 54:1876-1882), as well as safety and tolerability in pancreatic cancer patients at biologically active doses (O'Reilly et al., Journal of Clinical Oncology (2017); 35:4110-4110). A PDAC-targeting CAR was therefore constructed using the 5B1 scFv. Le^{A}-specific LBBz CARs directed effective cytotoxicity against multiple pancreatic cancer tumor lines expressing Le^{A}, but not Le^{A}-negative PC3 prostate cancer cells (FIGS. 13A-13C). Capan2 PDAC expresses an intermediate level of Le^{A} (FIG. 13B), and was selected for further experiments.

Low Dose RT Sensitizes Pancreatic Cancer to CAR T Cell Killing In Vitro and In Vivo

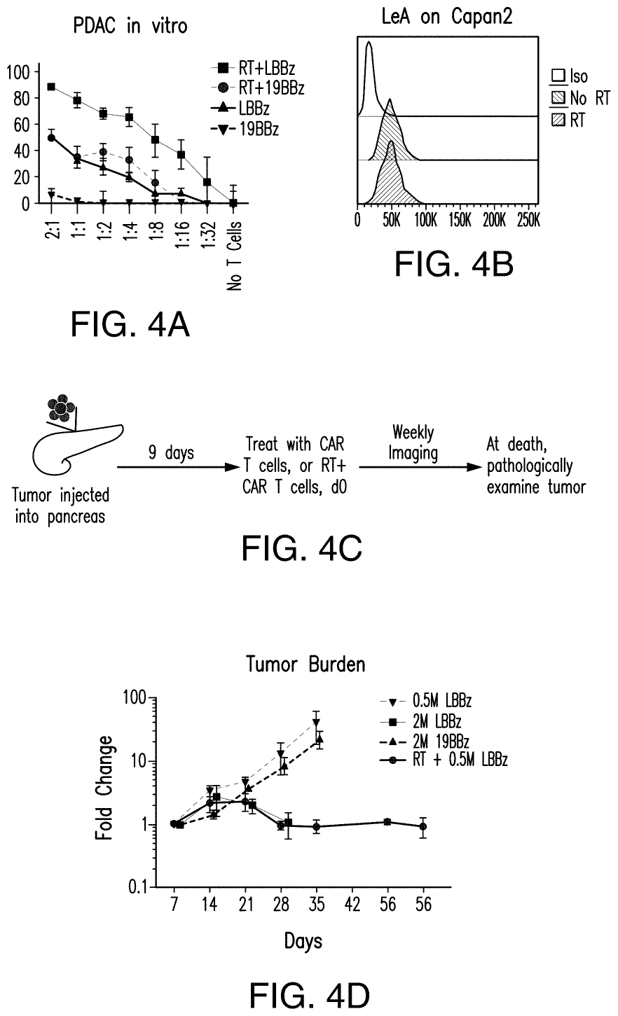

It was first questioned whether low-dose RT may have similar sensitizing effects in the solid PDAC tumor as observed in the ALL model. In vitro exposure of Capan2 to low-dose RT sensitized the tumor cells to Le^{A}-specific CARs (FIG. 4A), without altering Le^{A} expression (FIG. 4B), consistent with findings in the CD19 model (FIG. 1F). To test whether RT sensitization may be significant for this solid tumor in vivo, FACS-sorted LeA⁺ Capan2 tumor cells were injected orthotopically into the pancreas of NSG mice, allowed the tumors to establish for 9 days, and subsequently infused intravenously different doses of Le^{A} CAR T cells (FIG. 4C). A high dose of 2×10⁶ LBBz CAR T cells induced major tumor responses and delayed tumor progression, unlike the same dose of control 19BBz CART cells or a lower dose of 0.5×10⁶ LBBz CART cells (FIG. 4D). Sensitizing RT given one day before CAR T cell treatment significantly improved the ability of low dose CAR T cells to eradicate PDAC in these mice, similar to high dose CAR T cells without RT (FIG. 4D).

Ag⁻ Tumor Escape can be Controlled by DR5-Induced RT in Combination with CAR T Cells

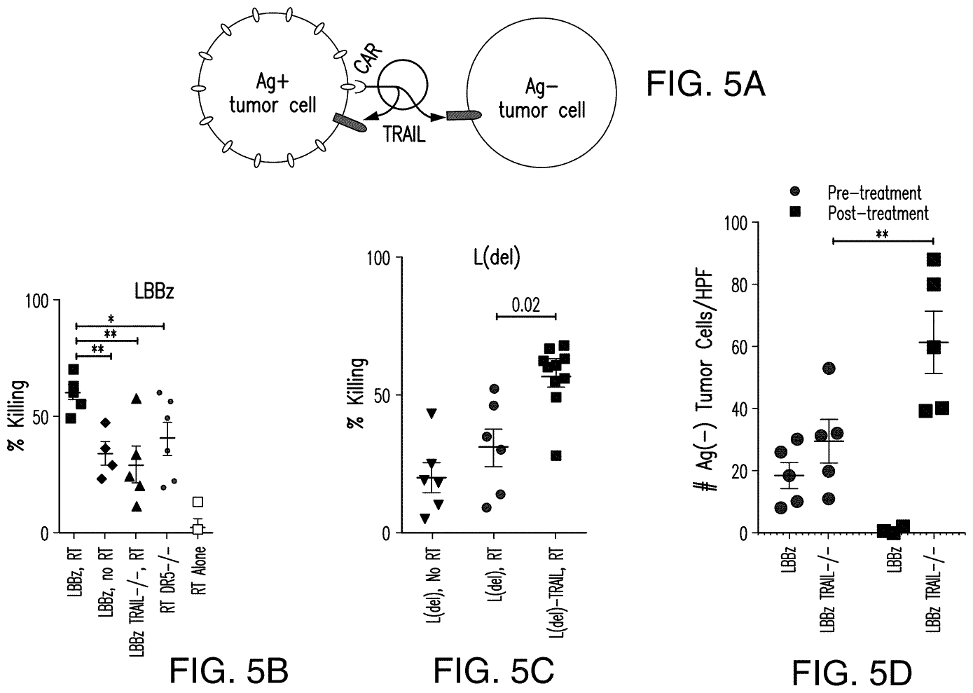

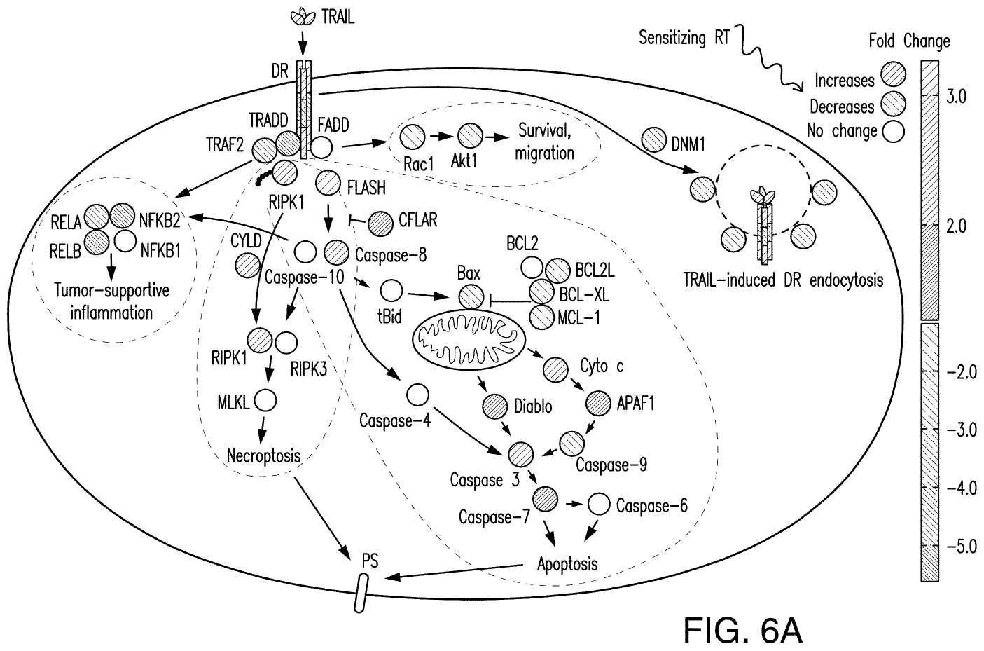

If DR pathway induction by low-dose RT is involved in sensitizing pancreatic cancer to CAR T cell mediated killing as it was found in Nalm6 ALL, it was hypothesized that expression of the CAR target on every tumor cell would not be required for complete tumor elimination through this mechanism (FIG. 5A). Whether Le^{A} heterogeneous PDAC could be controlled through the combined effect of RT and CAR T cells via TRAIL/DR5-mediated killing was then tested.

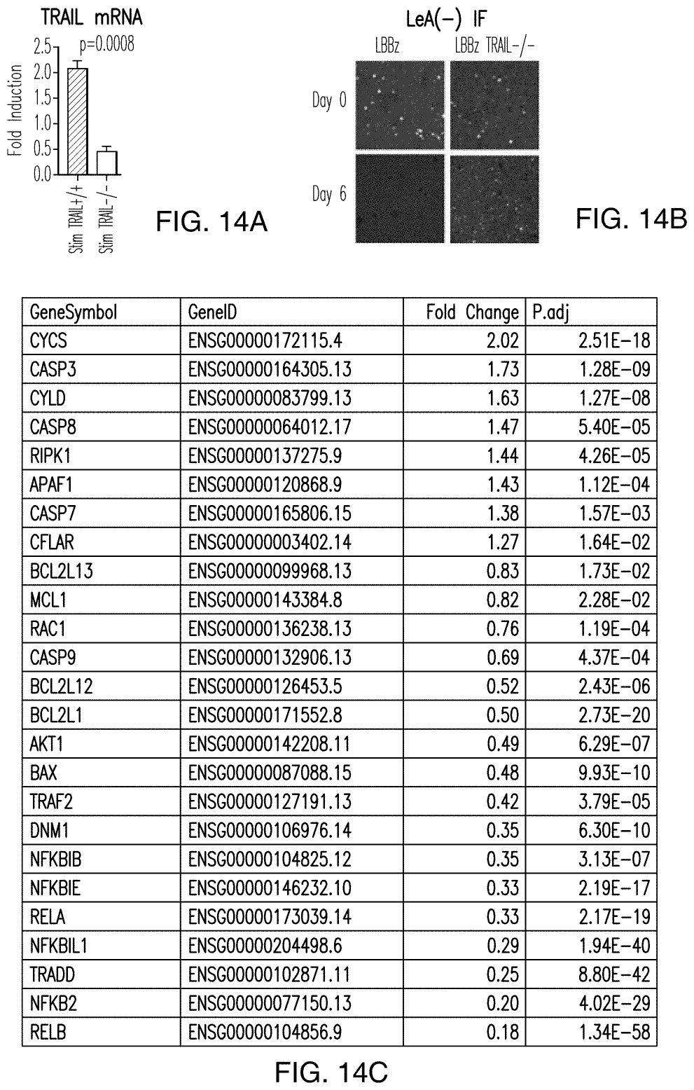

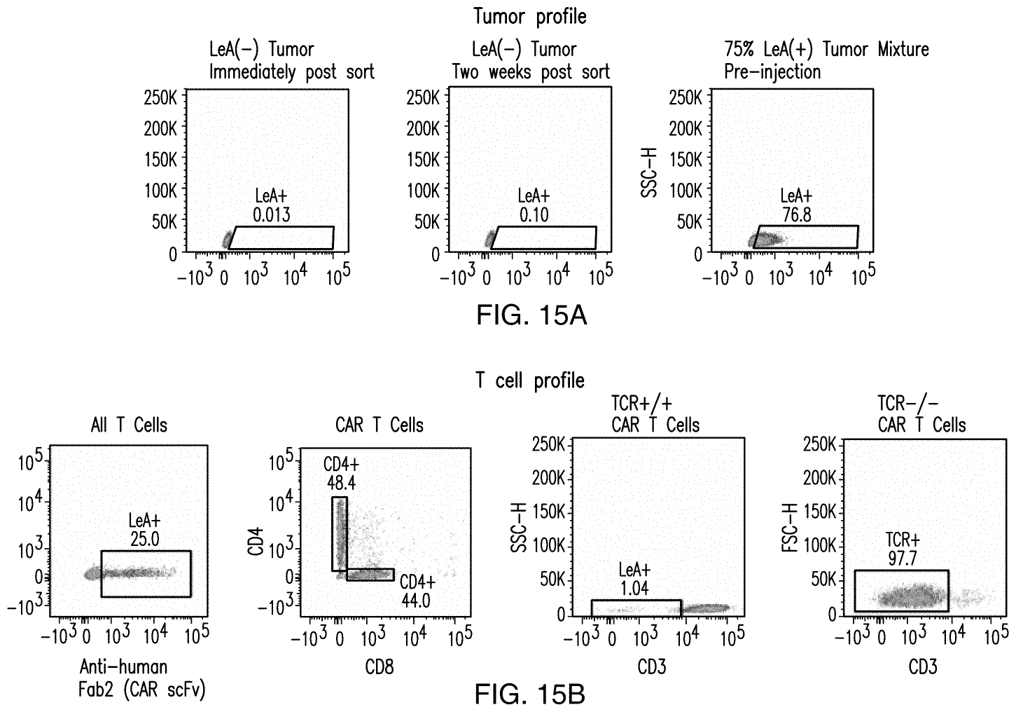

FACS sorted Ag⁻ cells were transduced with Firefly Luciferase (Luc), and remained stably antigen-negative over time (FIG. 15A). 75% Ag⁺ with 25% Ag⁻ Luc⁺ tumor cells was mixed, exposed to low dose or no RT, and incubated with CAR T cells in which TRAIL was disrupted by CRISPR (FIGS. 5A-5B, FIG. 14A). Using Luc activity to monitor Ag⁻ cell killing, it was found that wt CAR T cells on RT-exposed tumor cells produced the greatest magnitude of Ag⁻ tumor cell death, which was significantly reduced by the absence of TRAIL in the CAR T cell, or the absence of DR5 in the tumor (FIG. 5B). L(del) CAR T cells, which recognize the target cells but do not induce TRAIL, killed significantly more RT-sensitized Ag⁻ tumor cells if they were made to constitutively express TRAIL (FIG. 5C).

To further confirm the escaping Ag⁻ population is the original Ag⁻ population, and not a population that shed or lost its antigen due to another mechanism, RT-sensitized Ag⁻ PDAC tumor cells were labeled with CTV (blue) before mixing with sensitized Ag⁺ tumor cells and CAR T cells. It was found that TRAIL^{wt} CAR T cells are eventually able to eliminate both RT-sensitized Ag⁺ and Ag⁻ tumor cells in culture, while TRAIL^{-/-} CAR T cells allow outgrowth of the CTV-labeled Ag⁻ tumor cells over time (FIG. 5D, FIG. 14B).

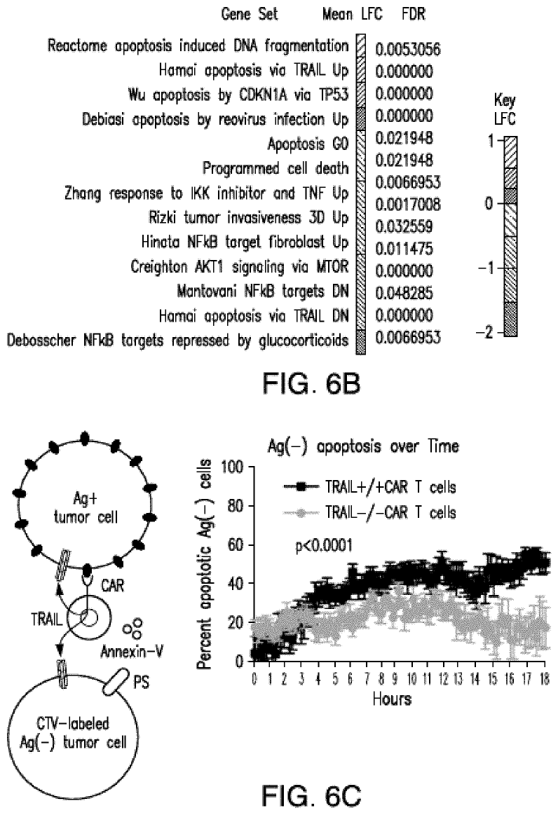

Sensitizing RT transcriptionally primes pancreatic cancer cells for TRAIL-induced death TRAIL can exert an anti-tumor role by inducing apoptosis and necroptosis, but under other circumstances TRAIL signaling in solid tumors can lead to pro-tumor effects including myeloid derived suppressor cell recruitment through NFkB activation-induced CCL2 production (Hartwig et al., Mol Cell (2017); 65:730-742 e735), or survival, invasion and metastasis through Rac1 and Akt activation within the tumor (von Karstedt et al., Cancer Cell (2015); 27:561-573). To better understand how pancreatic cancer cells might respond to increased TRAIL stimulation after sensitizing RT and CAR T cell therapy, known mediators of the described TRAIL signaling pathways was compiled and their gene expression changes were examined from RNAseq data before and after sensitizing RT in Capan2 cells (FIG. 6A). Many pathway mediators are also regulated post-translationally through cleavage, phosphorylation, ubiquitination, or other events, but gene expression analysis can provide general information regarding overall pathway activation states. Notably, the majority of individual members of both pro-tumor and anti-tumor mediators downstream of TRAIL were significantly altered by sensitizing RT. Pro-survival, migration, metastasis, and tumor-supportive inflammation TRAIL pathway members were almost uniformly downregulated, while pro-apoptotic molecules were overwhelmingly induced, suggesting TRAIL-mediated apoptosis may predominate relative to other pathways after sensitizing RT (FIG. 6A, FIG. 14C).

Statistically perturbed GSA pathways implicated in Akt signaling, NFkB signaling, and cell death were identified and charted. Consistent with the collection of individual gene changes, sets that ranked in the positive direction were pro-apoptotic, while Akt and NFkB pathways associated with survival, invasion, and pro-tumor inflammation were downregulated (FIG. 6B). GSA additionally found highly significant alignment with gene sets that specifically confer susceptibility to TRAIL-mediated apoptosis (Hamai et al., Oncogene (2006); 25:7618-7634). Sensitizing RT affected both the upregulated and downregulated gene sets, transcriptionally changing the cells from a TRAIL-resistant to a more TRAIL-sensitive apoptotic phenotype (429/492 genes concordantly upregulated, 114/128 concordantly downregulated, FDR<10⁻⁵ for each pathway) (FIG. 6B).

CAR-Activated T Cells Induce TRAIL-Mediated Apoptosis of Ag⁻ Tumor Cells

To obtain visual confirmation of direct Ag⁻ tumor apoptosis through activated CAR T cell-produced TRAIL in a heterogeneous tumor population, Ag⁻ tumor cells were RT-sensitized and labeled with Cell Trace Violet (CTV), and mixed with Ag⁺ cells and Annexin V-PE in the presence of either TRAIL^{wt} or TRAIL^{-/-} CAR T cells and monitored with live video microscopy. Automated quantification of Ag⁻ tumor cells undergoing apoptosis demonstrated TRAIL^{-/-} CAR T cells fail to induce Ag⁻ tumor apoptosis over time, while TRAIL^{wt} CAR T cell-treated cultures effect steady and significant Ag⁻ tumor cell apoptosis (p<0.0001, FIG. 6C).

Pancreatic Tumor Containing a Resistant Ag⁻ Population can be Eliminated In Vivo by CAR T Cells Following Sensitizing RT

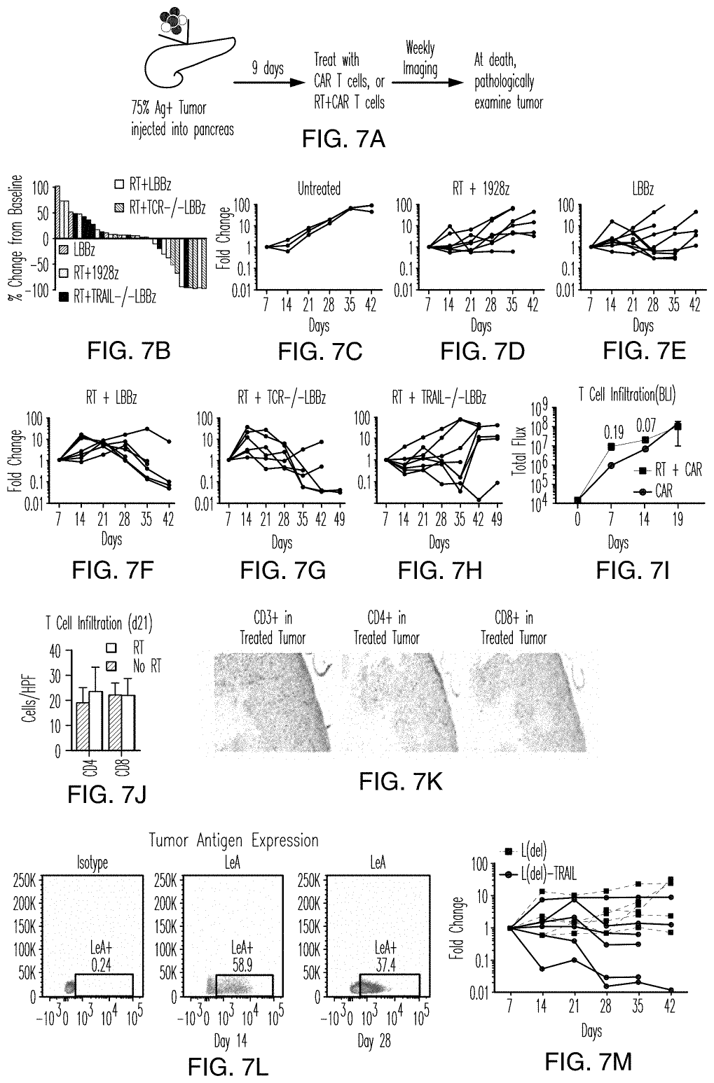

A mouse model was next established for the challenging but common clinical scenario of heterogeneous solid tumor partially devoid of target antigen. PDAC consisting of 25% Ag⁻ cells was established in the mouse pancreas, and nine days later treated with CAR T cells (FIG. 7A). CAR T cells that consistently eliminated Ag⁺ orthotopic PDAC were unable to completely eliminate any heterogeneous tumors (FIG. 7B-7E). Whether sensitizing RT afforded any meaningful benefit to heterogeneous tumor treated with CAR T cells in vivo was then tested. Mice with established heterogeneous PDAC, treated with sensitizing RT before CAR T cells achieved more CR and PR by imaging, autopsy exam, and pathology (FIGS. 7B and 7F). Since the major known

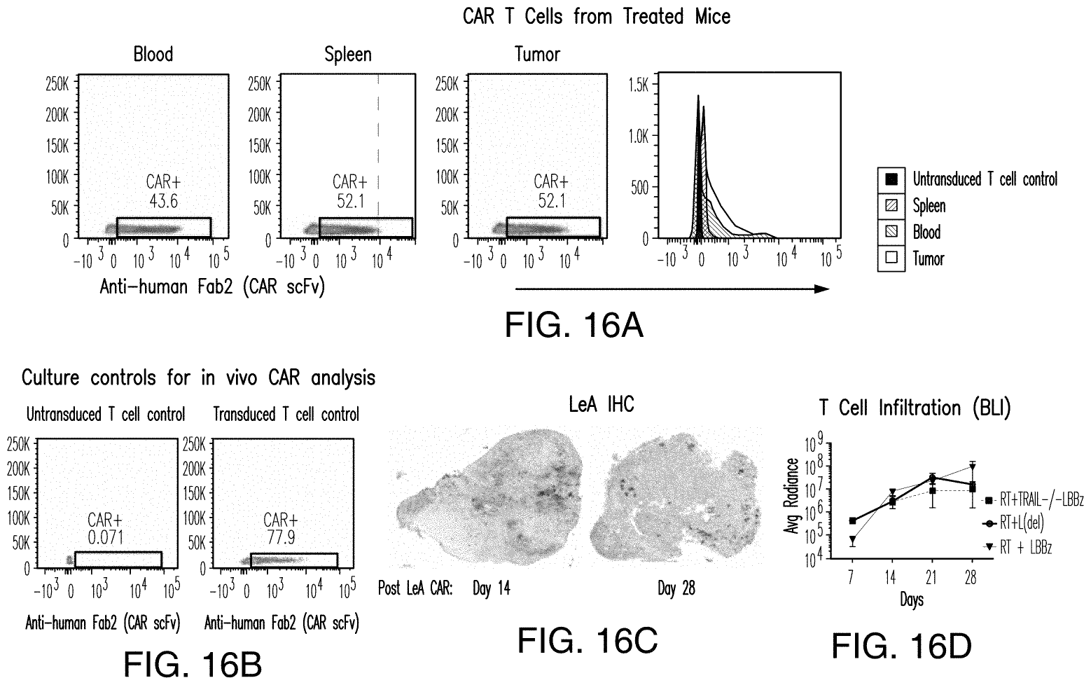

mechanism of CAR-independent T cell killing is through the T cell receptor (TCR), and RT can induce HLA expression on target cells, whether TCR-dependent tumor killing plays a significant role after sensitizing RT was investigated. CAR T cells lacking their TCR (TCR^{-/-}) (FIGS. 15A-15B) maintained the capacity to eliminate RT-sensitized heterogeneous tumor (FIG. 7G). RT initially resulted in moderately increased T cell accumulation within the tumor over the first two weeks (FIGS. 7I-7K). Despite significant tumor influx (FIG. 16D), TRAIL^{-/-} CAR T cells failed to consistently achieve a complete response in RT-sensitized tumor-bearing mice, demonstrated by both waterfall plot of response at the time of death (which occurred from either GVHD or tumor progression) (FIG. 7B), and weekly bioluminescence imaging (FIG. 7H). Mice with tumors that relapsed/progressed still harbored CAR T cells in the blood, spleen, and tumor as assessed by FACS, and exhibited significant T cells penetrating the tumor by IHC, but demonstrated outgrowth of Ag⁻ tumor cells (FIG. 7L, FIG. 16C).

To uncouple the effects of TRAIL and the CAR, RT-sensitized mice were treated with L(del) CAR T cells, which bind tumor but do not induce CAR cytotoxicity or TRAIL upon recognition, and L(del)-TRAIL CAR T cells, which bind tumor and constitutively express TRAIL but exert no CAR-mediated cytotoxicity. While the first strategy yielded no response despite local T cell accumulation (FIG. 7M, FIG. 16D), targeting constitutive TRAIL-expressing T cells to the tumor using the external CAR domain modestly increased the response rate (FIG. 7M).

Localized RT Effectively Conditions Tumor for Subsequent CAR T Cell Administration

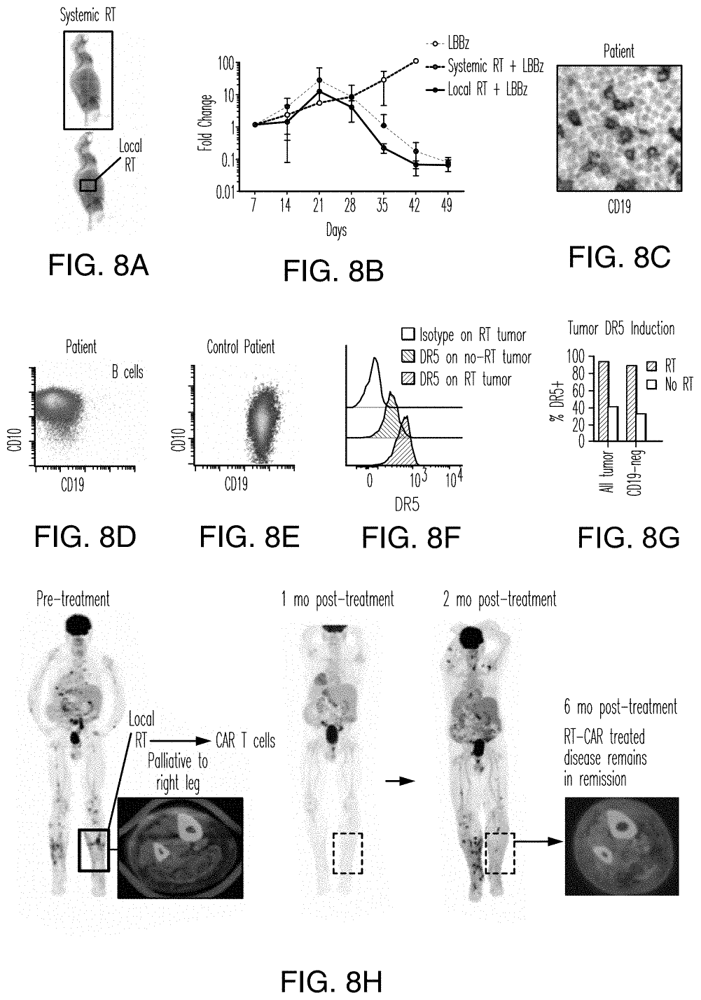

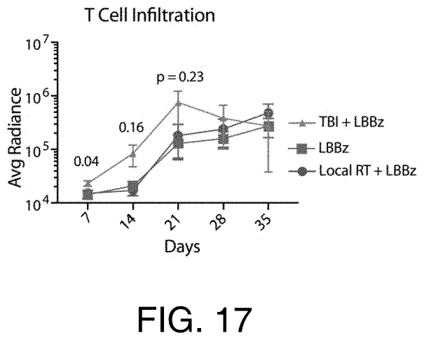

To determine whether systemic RT is required for CAR T cell sensitization, or if local RT to the tumor would suffice, mice harboring orthotopic PDAC were treated with RT to the whole body or only the pancreatic tumor, followed by CAR T cell administration (FIG. 8A). While total body RT-treated mice tended to have greater T cell tumor infiltration at early time points (FIG. 17), both strategies resulted in similar tumor responses (FIG. 8B). Thus, despite potentially different host effects between systemic and local low-dose RT, either approach effectively sensitizes heterogeneous tumor to CAR T cell killing.

Encouraging Risk/Benefit Ratio of RT and CAR T Cells in Patient with Heterogeneous Ag⁻ Tumor

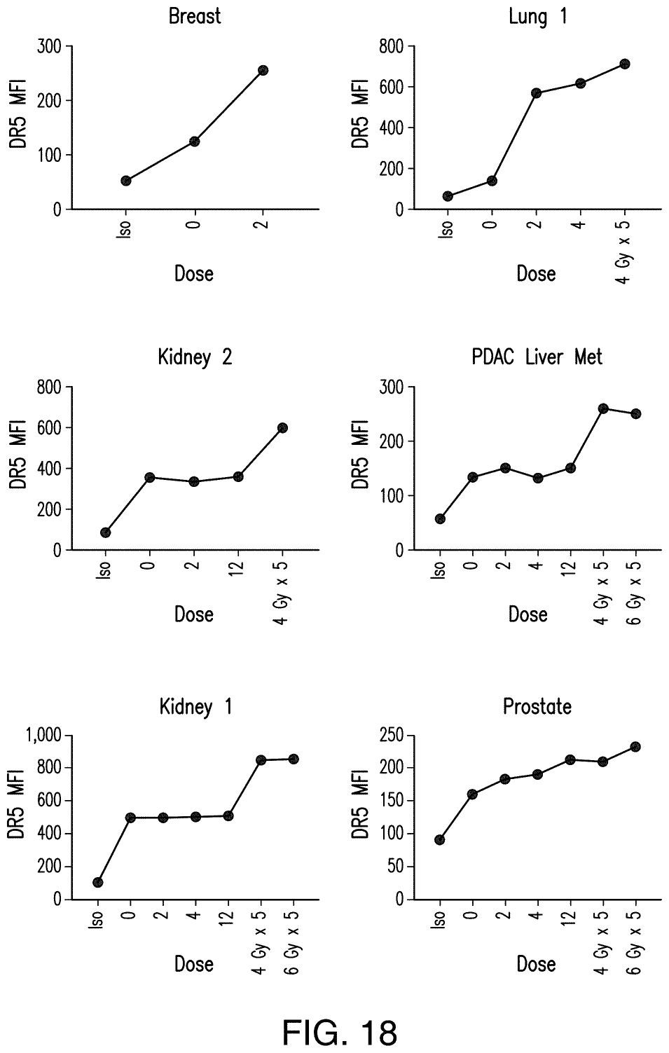

The applicability of RT sensitization to additional tumors is unknown, and experience combining RT with CAR T cells in patients is limited. To assess whether RT may affect the DR5 pathway in other tumors, patient-derived xenografts (PDX) were obtained from six different cancer patients. These cancers included non-small cell lung cancer, renal cell carcinoma (two patients), prostate cancer, pancreatic cancer, and breast cancer. Surface DR5 was upregulated in 5/6 of these patients' tumors after exposure to sublethal RT, although at different dose thresholds (FIG. 18). Expression was most consistently induced by the common palliative regimen of 4 Gy×5 (FIG. 18).

A patient with refractory diffuse large B cell lymphoma (DLBCL) bearing a large proportion of CD19⁻ tumor cells in the sampled tumor masses (FIGS. 8C-8E) presented for CD19 CAR therapy. The patient had painful disease infiltrating the skin of his lower legs, particularly on the right. His tumor, including the CD19⁻ population, induced DR5 expression ex vivo in response to 4 Gy×5 fractions (FIGS. 8F-8G). This dose of palliative RT was offered to his right leg, and he then received CD19 CAR T cells as planned. One month post-CAR T cells the patient had an excellent response, with Grade 1 cytokine release syndrome and no neurotoxicity or local adverse reaction. Two months post-CAR T cell infusion, tumor rebounded in prior and new locations, with the exception of the diseased area that received palliative RT before CAR T cells. While it is impossible to know how sensitive the patient's tumor would have been to

palliative RT alone, currently, one year after treatment, his antigen-heterogeneous tumor subjected to RT followed by CAR T cells remains in CR (FIG. 8H).

Discussion

The initial choice to target CD19 in B cell malignancies was largely driven by the elevated and relatively homogeneous expression of CD19 in leukemia and lymphoma and its confinement to the B cell lineage in normal tissues (Brentjens et al., Nat Med (2003); 9:279-286; Maher et al., Nat Biotechnol (2002); 20:70-75). Based on the remarkable complete remission rates of 70-90% in phase I ALL trial patients (Sadelain, J Clin Invest (2015); 125:3392-3400), the prospect of extending such success to a wide range of more common cancers was tantalizing. While CAR therapy has only recently begun to tackle solid tumors (Morello et al., Cancer Discov (2016); 6:133-146; Jindal et al., Med Oncol (2018); 35:87; DeSelm et al., J Surg Oncol (2017); 116:63-74), results have so far been modest with few occurrences of major responses (Louis et al., Blood (2011); 118:6050-6056; Brown et al., N Engl J Med (2016); 375:2561-2569). Escape and regrowth of antigen-negative tumor cells now being a well-documented mechanism of resistance to CAR therapy (Brown et al., N Engl J Med (2016); 375:2561-2569; Gardner et al., Blood (2016); 127:2406-2410; Jackson and Brentjiens, Cancer Discov (2015); 5:1238-1240), novel approaches are needed to enable CART cells to effectively prevent antigen escape.

An early approach to overcoming antigen escape from CAR T cells was to target two different antigens (Hegde et al., J Clin Invest (2016); 126:3036-3052). As it became more apparent that solid tumors often escape with far more than two antigenic variants, “armored CARs” were designed to secrete activating cytokines such as IL-18 (Avanzi et al., Cell Rep (2018); 23: 2130-2141), or to express an additional costimulatory receptor (Zhao et al., Cancer Cell (2015); 28:415-428) to stimulate neighboring T cells. Checkpoint inhibitor therapy has since been added to CAR T cells, aiming to reinvigorate both the CAR T cells and endogenous tumor-reactive T cells (Suarez et al., Oncotarget (2016); 7:34341-34355; Cherkassky et al., J Clin Invest (2016); 126:3130-3144). However, all of these approaches rely upon tumor cells to express a tumor-specific antigen that is recognized by either the CAR or the TCR; none of these approaches provide a mechanism by which an antigen-barren tumor cell can subsequently become recognized and eliminated.

The approach is unique because it delineates a mechanism by which a tumor cell devoid of antigen recognizable by a TCR or by the CAR can still be eliminated by a CAR T cell, in trans. This approach may be particularly beneficial in tumors with low mutational burden, where the probability of neoantigen presentation and recognition is otherwise very low.

Selectively activating a drug, including CAR T cells, within the tumor microenvironment is an appealing strategy to enhance efficacy or minimize off-target toxicity. While there are continuously advancing synthetic approaches to improving selectivity (Kloss et al., Nat Biotechnol (2013); 31:71-75; Roybal et al., Cell (2016); 164:770-779; Cho et al., Cell, (2018); 173:1426-1438), the spatial and temporal specificity achieved here relies upon a physiologic response of the CAR T cell. The observation that TRAIL is induced in CAR T cells after tumor encounter ensures active and maximal production within the tumor microenvironment. The ability to induce sensitivity to TRAIL-mediated death on Ag⁺ and Ag⁻ tumor cells through targeted RT provides a window of opportunity to enhance site-specific CAR T cell efficacy against heterogeneous tumor. The effect of this interaction has multiple implications. In the case of systemic leukemia, low dose total body irradiation effectively sensitizes tumor cells to CAR T cell killing and results in CAR-mediated elimination of systemic disease at a much lower CAR T cell dose, potentially reducing the risk for cytokine release syndrome while increasing the efficacy. In the case of localized orthotopic pancreatic tumor, it was found that both systemic and localized RT sensitize the tumor to CAR T cell killing. Most importantly, in antigen-heterogeneous pancreatic cancer, it was shown that Ag⁻ tumor cells that would otherwise escape CAR recognition can be eliminated by CAR T cells after sensitizing RT in vivo.

The early observation that tumor cells are highly sensitive to TRAIL-induced apoptosis relative to normal cells (Walczak et al., Nat Med (1999); 5:157-163) generated enthusiasm for recombinant TRAIL or agonistic TRAIL receptor-based therapies. Unfortunately, this therapy has encountered multiple limitations, including short half-life of TRAIL protein (Ichikawa et al., Nat Med (2001); 7: 954-960), reduced apoptotic ability of bivalent antibody (Wajant, Cell Death Differ (2015); 22:1727-1741), limited local

tumor penetration when administered systemically, and downstream resistance to apoptosis through tumor gene expression changes (Ichikawa et al., Nat Med (2001); 7: 954-960). CAR T cells as the source of TRAIL offer several potential advantages, such as concentrated synthesis within the tumor, continuous production as long as tumor is present, and supply of native trimeric protein rather than potentially less apoptotic bivalent antibody (Wajant, Cell Death Differ (2015); 22:1727-1741). Since low dose RT affected a signature of over 500 genes implicated in TRAIL apoptotic susceptibility, and TRAIL knockout in the T cell generally exerted a stronger phenotype than DR5 knockout in the pancreatic tumor, the critical mediators of TRAIL sensitivity after RT in different tumor types is a subject of further investigation.

RT is currently used at some point in the treatment of roughly half of metastatic cancer patients for palliation, and is commonly utilized in almost all non-metastatic cancer types as an alternative, or an addition to surgery to increase local control (Miller et al., CA Cancer J Clin (2016); 66:271-289). Implementing CARs into RT-treatment regimens may further increase local and systemic control. As CAR T cells become more prevalent, so will the coincidental co-administration of RT and CAR T cells. The data suggests the timing of delivery of these two therapies is critically impactful, and warrants coordination among disease management teams.

Several other forms of immunotherapy are commonly combined with RT under certain circumstances. An “immunogenic,” ablative high dose of radiation induces tumor death and in some contexts leads to increased antigen presentation, subsequent T cell activation, and potentially an “abscopal” or secondary immune response against unirradiated tumor (Spiotto et al., Sci Immunol (2016); 1). Due to the infrequency of the abscopal effect in clinical practice, predictably harnessing this phenomenon remains an active area of investigation. Unlike endogenous T cells, CAR T cells do not rely on antigen presentation, and unless radiation induces expression of the particular CAR target molecule (Weiss et al., Cancer Res (2018); 78:1031-1043), it is not intuitive whether radiation may have an immunogenic, immunosuppressive, or irrelevant effect on CAR T cell therapy. A fundamentally different type of “immunogenic radiation” in the context of CAR T cell therapy was described: one by which a sublethal, low dose of radiation locally sensitizes tumor to CAR T cell killing in trans. Unlike its ablative counterpart, sensitizing radiation is not limited by location or size of disease, and given the much lower dose may be applied to wider areas for patients with diffuse metastases, with less concern for RT-related side effects.

A patient with heterogeneous tumor that also induced DR5 upon palliative RT exposure, treated as such before CAR T cell therapy exhibited results consistent with the mouse data. Although this clinical correlate aligns with the animal findings, it does not test the hypothesis. In particular, the effect of RT alone on the lasting complete response of his heterogeneous tumor cannot be ignored. However, the administered radiation dose was unable to eliminate his tumor ex vivo, consistent with it being roughly half the standard locally curative dose of >45 Gy for gross disease in this type of aggressive lymphoma (Ng et al., International journal of radiation oncology, biology, physics (2018); 100:652-669). A clinical trial incorporating RT with CAR T cells is planned to assess the effect on clonal antigen heterogeneity, the safety of RT conditioning, and systemic effects of local RT on CAR T cell-mediated disease response.

The findings support the concept that multimodality CAR therapy with RT conditioning may improve responses in both hematologic and solid tumors. Most importantly, a mechanistic platform was provided by which engineered T cells may be further enhanced to eliminate clonally heterogeneous solid tumors.

Materials and Methods

Cell Culture

NALM6 expressing firefly luciferase-GFP were described previously (Zhao et al., Cancer Cell (2015); 28:415-428). 293T cell line, H29 and retroviral packaging cell lines were cultured in DMEM supplemented with 10% FCS. CD19+ NIH-3T3 was described previously (Zhao et al., Cancer Cell (2015); 28:415-428). Capan-2 cell were generously provided by Jason S. Lewis (MSKCC) and grown in RPMI supplemented with 10% FCS.

Cells were tested for Mycoplasma using the MycoAlert Mycoplasma Detection Kit (Lonza) prior to injection into the animals.

Buffy coats from healthy volunteer donors were obtained from the New York Blood Center. Peripheral blood mononuclear cells were isolated by density gradient centrifugation, and cells were then stimulated with PHA (Sigma) and cultured as previously described (Zhao et al., Cancer Cell (2015); 28:415-428).

Radiation

Radiation dose: All experiments using Nalm6 ALL tumor cells used 1.8 Gy, and all experiments using PDAC used 2 Gy, unless specifically otherwise specified. For in vitro RT studies, all RT sensitization experiments were performed with RT given to the tumor cells two days prior to tumor analysis or coculture with T cells, unless specified otherwise.

Radiation method: Local RT to the pancreas was performed by identifying the pancreatic tumor using intraperitoneal contrast and cone beam CT imaging on an X-Rad 225Cx machine, which combines high-accuracy cone beam CT imaging with 3D image guided radiation treatment under general anesthesia. Local RT was delivered using either anterior-posterior, or anterior-posterior and lateral beams. Experiments requiring less target precision (total body RT) were performed using the small animal irradiator with open jaws in the AP direction.

Flow Cytometry

Fluorochrome-conjugated antibodies to CD3 (UCHT1), CD4 (S3.5), CD8 (3B5), DR5 (DJR2-4, PE-conjugated, BioLegend), CD95 (DX2, PE-Cy7-conjugated, BD Biosciences), LeA (7LE, AF405-conjugated, Novus), CD19 (SJ25C1), 41BBL (5F4), and Granzyme B (FGB12, Invitrogen) were used. Alexa 647-conjugated goat anti-human F(ab)2 (ThermoFisher) was used to detect CARs. Flow cytometry was performed on a BD LSRII and data analyzed with FlowJo software Ver. 9.5.2 (TreeStar). Fc Receptor Binding Inhibitor Antibody Human (eBioscience) was used to block Fc receptors. In some cases, CountBright beads (Invitrogen) were added to samples to count cell numbers.

TRAIL Measurements

For RNA and ELISA experiments, 1928z CAR T cells were exposed to 3T3 target cells expressing CD19 for 4 hours followed by removal and monoculture of T cells, which were replated in new media daily. Cells were removed and analyzed for TRAIL mRNA expression at given time points, and media was collected at the end of each day for TRAIL ELISA (MyBiosource MBS335491). qPCR was performed using the TaqMan system (ThermoFisher), using primers Hs00921974 (TRAIL), Hs00366278 (DR5), and Hs04194366 (RPL13A housekeeping).

RNA Extraction and Real-Time Quantitative PCR

Total RNA was extracted from T cells by using the RNeasy kit (QIAGEN) combined with QIAshredder (QIAGEN), following the manufacturer's instructions. RNA concentration and quality were assessed by UV spectroscopy using the NanoDrop spectrophotometer (Themo Fisher Scientific). One hundred to 200 ng total RNA were used to prepare cDNA using the SuperScript III First-Strand Synthesis SuperMix (Invitrogen), with a 1:1 volume ratio of random hexamers and oligo dT. Completed cDNA synthesis reactions were treated with 2U RNase H for

20 min at 37° C. Quantitative PCR was performed using the ABsolute Blue qPCR SYBR Green Low ROX Mix. PCR assays were run on the QuantStudio™ 7 Flex System, and Ct values were obtained with the QuantStudio Real-Time PCR software. Relative changes in gene expression were analysed with the 2^{AACt} method.

Vector Constructs

The 1928ζ and 19BBζ CARs, which comprise the SJ25C1 CD19-specific scFv (Brentjens et al., Nat Med (2003); 9:279-286), have been previously described (Weiss et al., Cancer Res (2018); 78:1031-1043). LBBz and L28z were constructed by replacing the CD19-specific scFv with the human 5B1 scFv targeting Le^{A}. All constructs were designed to express Gaussia Luciferase for T cell imaging as previously described (Santos et al., Nat Med (2009); 15:338-344). L(del) and 19(del) mutants were created by removing the intracellular costimulatory and signaling domains from the specified construct, while retaining the extracellular and transmembrane portion. Constructs expressing TRAIL were created by adding the TRAIL cDNA sequence following the specified CAR and a P2A sequence.

Retroviral Production and Transduction

Plasmids encoding the SFG γ-retroviral (RV) vector (Riviere et al., Proc Natl Acad Sci USA (1995); 92:6733-6737) were prepared as previously described (Maher et al., Nat Biotechnol (2002); 20:70-75). VSV-G pseudotyped retroviral supernatants derived from transduced gpg29 fibroblasts (H29) were used to construct stable retroviral-producing cell lines as previously described (Gallardo et al., Blood (1997); 90:952957). T cells were transduced by centrifugation on Retronectin (Takara)-coated plates. In T cell knockout studies, CAR transduction was performed directly after Cas9/gRNA electroporation as described (Eyquem et al., Nature (2017); 543, 113-117).

Cytotoxic T Lymphocyte Assays (CTLs)

CTLs using 100% Ag+ tumor cells: The cytotoxicity of T cells transduced with a CAR was determined by standard luciferase based assays. Tumor cells expressing firefly luciferase-GFP served as target cells. The effector and tumor cells were co-cultured at indicated E/T ration in the black-walled 96 or 384 well plates in triplicate manner. Target cells alone were plated at the same cell density to determine the baseline luciferase expression (no T cell control). 18 hr later, luciferase substrate (Bright-Glo, Promega) was directly added to each well. Emitted light was measured by luminescence plate reader or Xenogen IVIS Imaging System (Xenogen) with Living Image software (Xenogen) for acquisition of imaging data sets. Lysis was determined as [1−(RLUsample)/(RLUmax)]×100. Assays were performed using CAR T cells transduced within the previous week.

CTLs using 75% Ag+, 25% Ag− tumor cells: In experiments involving pre-stimulated CAR T cells, all CAR T cells were grown for 10-12 days at a constant concentration of 1 million cells/ml in the presence of 20 U/ml IL-2, reconstituted every other day. CAR T cells were stimulated three days before CTL by adding the CAR T cells to adherent cells containing the target antigen (Le^{A}+ Capan2). CAR T cells were then cocultured with RT-sensitized 75% Ag⁺ Capan2 PDAC at an E:T ratio of 1:3 in 48 well plates, in which only the Ag− cells expressed luciferase. Percent killing relative to no treatment controls was determined at the pre-specified time points of 4 days for LBBz CAR T cell cultures, and 5 days for L(del) CAR T cell cultures.

In all cytotoxicity assays where RT was used, tumor cells (Nalm6 or Capan2) were given RT, grown for two days in culture, then live cells were incubated with CAR T cells.

Video Microscopy

Ag⁻ cells labeled with CTV (CellTrace Violet, Fisher C34571) were mixed with

unlabeled Ag⁺ cells at a ratio of 75% LeA⁺, in addition to CAR T cells in 8-well microscopy slides, and Annexin-V 595 (Fisher A13203). Confocal images were acquired every 7 minutes over 18 hours in culture at optimal imaging parameters with a LSM 880 Confocal Microscope (Carl Zeiss). Data was 3D-rendered and visualized using Imaris (Bitplane). Percent killing of LeA⁻ cells was determined at every time point using a custom macro made in ImageJ/FIJI (NIH), which automatically quantified total Ag⁻ cells (blue cells) and dead/dying Ag⁻ cells (double red and blue positive).

Gene Disruption

48 h after initiating T-cell activation, the cells were transfected by electrotransfer of Cas9 mRNA and gRNA using an AgilePulse MAX system (Harvard Apparatus). 3×10⁶ cells were mixed with 5 jig of Cas9 and 5 jig of gRNA into a 0.2 cm cuvette. Following electroporation cells were diluted into culture medium and incubated at 37° C., 5% CO2. To obtain TCR-negative T cells, TCR-positive T cells were removed 3-5 days after gRNA transfection using magnetic biotin-anti-TCRαβ and anti-biotin microbeads and LS columns (Miltenyi Biotech). To obtain TRAIL-negative cells, TRAIL-positive T cells were removed using magnetic PE-anti-TRAIL (R&D, FAB687P) and anti-PE microbeads in LS columns (Miltenyi Biotech). To obtain DR5-negative cells, FACS sorting was performed using PE-anti-DR5 staining.

For TCR knockout, a gRNA that targets a sequence in the first exon of the constant chain of the TCRα gene (TRAC) that is required for the TCRα and β assembly and addressing to the cell-surface was used as previously described (41). DR5, TRAIL, and FAS knockout was performed using synthetic modified gRNA kits (Synthego). Guide RNAs were reconstituted at 1 jig ji1⁻¹ in cytoporation T Buffer (Harvard Apparatus). Cas9 mRNA was synthesized by TriLink Biotechnologies.

Mouse Systemic Nalm6 Tumor Model

8- to 12-week-old NOD/SCID/IL-2Rγ-null (NSG) male mice (Jackson Laboratory) was used under a protocol approved by the MSKCC Institutional Animal Care and Use Committee. Mice were inoculated with 0.5×10⁶ FFLuc-GFP NALM-6 cells by tail vein injection, followed by 1.8 Gy total body RT, and 10⁴ CAR T cells injected four days later. Mice were randomized to treatment and blinded to personnel performing treatment injections and tumor assessment by use of numbers. Bioluminescence imaging used the Xenogen IVIS Imaging System (Xenogen) with Living Image software (Xenogen) for acquisition of imaging datasets.

For RT timing experiments, mice were treated in the same manner except CAR T cells were delivered either the day after RT, or 7 days after RT. All animal care was in accordance with institutional guidelines.

Pancreatic Cancer Tumor Model

8- to 12-week-old NSG mice (Jackson Laboratory) were used under a protocol approved by the MSKCC Institutional Animal Care and Use Committee. Specified ratios of LeA+ and LeA− FACS-sorted Capan2 PDAC tumor cells were injected into the pancreas of NSG mice after surgically opening the mice and exposing the pancreas under IRB-approved mouse protocol. 75,000 tumor cells were injected per mouse, in 50% matrigel. Mice were randomized to treatment and treatment groups were blinded to personnel performing treatment and tumor assessment. Tumor established in the pancreas for 9 days, then mice were treated with RT followed by T cells. Tumor volume was measured by Bioluminescence Imaging (BLI) using retro-orbital D-luciferin injection followed by IVIS imaging. Tumor burden for each mouse was expressed over time relative to that mouse's baseline tumor BLI at the beginning of treatment.

T Cell Imaging

CAR T cells containing Gaussia Luciferase were imaged using coelenterazine (3031-10 Coelenterazine-SOL in vivo, Nanolight), injected retro-orbitally.

Transcriptome Analysis

Cells were lysed in Trizol LS (Invitrogen) and then submitted to the Integrated Genomics Operation at MSKCC for RNA extraction. After ribogreen quantification and quality control on bioAnalyser, 500 ng of total RNA underwent library preparation using the Truseq Stranded Total RNA library preparation chemistry (Illumina), with 6 cycles of PCR. Samples were barcoded and run on a Hiseq 2500 1T in a 50 bp/50 bp Paired end run, using the TruSeq SBS Kit v3 (Illumina). An average of 51 million paired reads were generated per sample and the percent of mRNA bases was 58% on average.

The output FASTQ data files were mapped to the target genome using the rnaStar aligner which maps reads genomically and resolves reads across splice junctions. 2 pass mapping method outlined in which the reads are mapped twice. The first mapping pass uses a list of known annotated junctions from Ensemble. Novel junctions found in the first pass are then added to the known junctions and a second mapping pass is done (on the second pass the RemoveNoncanoncial flag is used). After mapping, the output SAM files were post processed using the PICARD tools to: add read groups, AddOrReplaceReadGroups which in additional sorts the file and coverts it to the compressed BAM format. The expression count matrix was then computed from the mapped reads using HTSeq (www-huber.embl.de) and one of several possible gene model databases. The raw count matrix generated by HTSeq are then be processed using the R/Bioconductor package DESeq (www-huber.embl.de) which is used to both normalize the full dataset and analyze differential expression between sample groups.

For GSA the Bioconductor package PIANO was used (bioconductor.org). The precise call is: gsa.res<-runGSA(fc,geneSetStat=“mean”,gsc=gsc, gsSizeLim=c(min.gns,max.gns), nPerm=nPerm), where fc==foldChange, min.gns==5, max.gns==1000, nPerm==1e4. For GeneSets the MSigDb from the Broad was used (software.broadinstitute.org). The following collections were used: “c1.all.v4.0.symbols.gmt”, “c2.all.v4.0.symbols.gmt”, “c3.all.v4.0.symbols.gmt”, “c5-1.all.v4.0.symbols.gmt”, “c6-1.all.v4.0.symbols.gmt”, “c7.all.v4.0.symbols.gmt”.

Statistics

All experimental data are presented as mean±s.e.m. No statistical methods were used to predetermine sample size. Groups were compared using unpaired, two-tailed t-test. Statistical analysis was performed on GraphPad Prism 7 software.

### EMBODIMENTS OF THE PRESENTLY DISCLOSED SUBJECT MATTER

From the foregoing description, it will be apparent that variations and modifications may be made to the the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunoresponsive cell comprising:
(a) an antigen-recognizing receptor that binds to an antigen, and
(b) an exogenous TRAIL polypeptide.

2. An immunoresponsive cell comprising:
(a) an antigen-recognizing receptor that binds to an antigen, and
(b) a modified enhancer or promoter at an endogenous TRAIL gene locus.

3. A composition comprising an effective amount of a cell of claim 1.

4. A method of reducing tumor burden in a subject, treating and/or preventing a neoplasm in a subject, and/or lengthening survival of a subject having a neoplasm, the method comprising administering to the subject an effective amount of immunoresponsive cells of claim 1.

5. A method for producing an antigen-specific immunoresponsive cell, the method comprising introducing into an immunoresponsive cell (a) a first nucleic acid sequence encoding an antigen-recognizing receptor that binds to an antigen; and (b) a second nucleic sequence encoding an exogenous TRAIL polypeptide, wherein each of the first and second nucleic acid sequence optionally operably linked to a promoter element.

6. A nucleic acid composition comprising (a) a first nucleic acid sequence encoding the antigen-recognizing receptor of claim 1, and (b) a second nucleic acid sequence encoding the exogenous TRAIL polypeptide of claim 1.

7. A vector comprising the nucleic acid composition of claim 6.

8. A kit comprising an immunoresponsive cell of claim 1.

9. A method of reducing tumor burden or treating and/or preventing a neoplasm in a subject, the method comprising:
i) administering to the subject a radiation therapy; and
ii) administering to the subject an effective amount of immunoresponsive cells or a pharmaceutical composition comprising thereof.

10. The method of claim 9, wherein the method reduces the number of tumor cells, reduces tumor size, and/or eradicates the tumor in the subject.

11. The method of claim 9, wherein the radiation therapy is administered in a total dose of no more than about 30 Gy.

12. The method of claim 9, wherein the radiation therapy is administered in a total dose of between about 1 Gy and about 20 Gy.

13. The method of claim 9, wherein the radiation therapy is administered no later than about 20 days prior to the administration of the immunoresponsive cells.

14. The method of claim 9, wherein the radiation therapy is administered no later than about 10 days prior to the administration of the immunoresponsive cells.

15. The method of claim 9, wherein the radiation therapy is an external beam radiation therapy, a brachytherapy or a systemic radioisotope therapy.

16. The method of claim 9, wherein the immunoresponsive cell comprises an antigen recognizing receptor.

17. The method of claim 16, wherein the antigen-recognizing receptor is a chimeric antigen receptor (CAR) or a T-cell receptor (TCR).

18. The method of claim 17, wherein the antigen-recognizing receptor is a chimeric antigen receptor (CAR).

19. The method of claim 9, wherein the immunoresponsive cell comprises (a) an antigen-recognizing receptor that binds to an antigen, and (b) an exogenous TRAIL polypeptide.

20. The method of claim 9, wherein the immunoresponsive cell comprises (a) an antigen-recognizing receptor that binds to an antigen, and (b) a modified enhancer or promoter at an endogenous TRAIL gene locus.


SEQUENCE LISTING (63 sequences)

SEQ ID NO: 1 - protein, 15 aa, Artificial Sequence
  Description of Artificial Sequence Synthetic peptide
GGGGSGGGGSGGGGS

SEQ ID NO: 2 - protein, 20 aa, Homo sapiens
MYRMQLLSCIALSLALVTNS

SEQ ID NO: 3 - protein, 20 aa, Mus sp.
MYSMQLASCVTLTLVLLVNS

SEQ ID NO: 4 - protein, 20 aa, Homo sapiens
METPAQLLFLLLLWLPDTTG

SEQ ID NO: 5 - protein, 20 aa, Mus sp.
METDTLLLWVLLLWVPGSTG

SEQ ID NO: 6 - protein, 21 aa, Homo sapiens
MALPVTALLLPLALLLHAARP

SEQ ID NO: 7 - protein, 18 aa, Homo sapiens
MALPVTALLLPLALLLHA

SEQ ID NO: 8 - protein, 16 aa, Homo sapiens
MKWVTFISLLFSSAYS

SEQ ID NO: 9 - protein, 30 aa, Homo sapiens
MDSKGSSQKGSRLLLLLVVSNLLLCQGVVS

SEQ ID NO: 10 - protein, 263 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 11 - DNA, 789 bp, Artificial Sequence
  Description of Artificial Sequence Synthetic polynucleotide
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 12 - protein, 122 aa, Artificial Sequence
  Description of Artificial Sequence Synthetic polypeptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 13 - protein, 108 aa, Artificial Sequence
  Description of Artificial Sequence Synthetic polypeptide
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 14 - protein, 7 aa, Artificial Sequence
  Description of Artificial Sequence Synthetic peptide
GYAFSSY

SEQ ID NO: 15 - protein, 6 aa, Artificial Sequence
  Description of Artificial Sequence Synthetic peptide
YPGDGD

SEQ ID NO: 16 - protein, 13 aa, Artificial Sequence
  Description of Artificial Sequence Synthetic peptide
KTISSVVDFYFDY

SEQ ID NO: 17 - protein, 11 aa, Artificial Sequence
  Description of Artificial Sequence Synthetic peptide
KASQNVGTNVA

SEQ ID NO: 18 - protein, 7 aa, Artificial Sequence
  Description of Artificial Sequence Synthetic peptide
SATYRNS

SEQ ID NO: 19 - protein, 9 aa, Artificial Sequence
  Description of Artificial Sequence Synthetic peptide
QQYNRYPYT

SEQ ID NO: 20 - DNA, 21 bp, Artificial Sequence
  Description of Artificial Sequence Synthetic oligonucleotide
ggctatgcattcagtagctac

SEQ ID NO: 21 - DNA, 18 bp, Artificial Sequence
  Description of Artificial Sequence Synthetic oligonucleotide
tatcctggagatggtgat

SEQ ID NO: 22 - DNA, 39 bp, Artificial Sequence
  Description of Artificial Sequence Synthetic oligonucleotide
aagaccattagttcggtagtagatttctactttgactac

SEQ ID NO: 23 - DNA, 33 bp, Artificial Sequence
  Description of Artificial Sequence Synthetic oligonucleotide
aaggccagtcagaatgtgggtactaatgtagcc

SEQ ID NO: 24 - DNA, 21 bp, Artificial Sequence
  Description of Artificial Sequence Synthetic oligonucleotide
tcggcaacctaccggaacagt

SEQ ID NO: 25 - DNA, 27 bp, Artificial Sequence
  Description of Artificial Sequence Synthetic oligonucleotide
caacaatataacaggtatccgtacacg

SEQ ID NO: 26 - DNA, 366 bp, Artificial Sequence
  Description of Artificial Sequence Synthetic polynucleotide
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctca

SEQ ID NO: 27 - DNA, 324 bp, Artificial Sequence
  Description of Artificial Sequence Synthetic polynucleotide
gacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 28 - protein, 142 aa, Artificial Sequence
  Description of Artificial Sequence Synthetic polypeptide
MEFGLSWLFLVAILKGVQCQVQLVESGGGSVQPGRSLRLSCEASGFTFEAYAMHWVRQPPGKGLEWVSSINWNSGRIAYADSVKGRFTISRDNARNSLYLQMNSLRLEDTAFYYCAKDIRRFSTGGAEFEYWGQGTLVTVSS

SEQ ID NO: 29 - protein, 130 aa, Artificial Sequence
  Description of Artificial Sequence Synthetic polypeptide
MAGFPLLLTLLTHCAGSWAQSVLTQPPSASGTPGQRVTISCSGSSSNIGSNFVYWYQQLPGTAPKLLIYRNNQRPSGVPDRFSGSRSGTSASLAISGLRSEDEADYYCAAWDDSLGGHYVFGTGTKVTVL

SEQ ID NO: 30 - protein, 8 aa, Artificial Sequence
  Description of Artificial Sequence Synthetic peptide
GFTFEAYA

SEQ ID NO: 31 - protein, 8 aa, Artificial Sequence
  Description of Artificial Sequence Synthetic peptide
INWNSGRI

SEQ ID NO: 32 - protein, 16 aa, Artificial Sequence
  Description of Artificial Sequence Synthetic peptide
AKDIRRFSTGGAEFEY

SEQ ID NO: 33 - protein, 8 aa, Artificial Sequence
  Description of Artificial Sequence Synthetic peptide
SSNIGSNF

SEQ ID NO: 34 - protein, 3 aa, Artificial Sequence
  Description of Artificial Sequence Synthetic peptide
RNN

SEQ ID NO: 35 - protein, 12 aa, Artificial Sequence
  Description of Artificial Sequence Synthetic peptide
AAWDDSLGGHYV

SEQ ID NO: 36 - DNA, 426 bp, Artificial Sequence
  Description of Artificial Sequence Synthetic polynucleotide
atggagtttgggctgagctggctttttcttgtggctattttaaaaggcgtacagtgccaggtgcagctggtggagtctgggggaggctcggtgcagcctggcaggtccctgagactctcctgtgaagcctctggattcacctttgaggcctatgccatgcactgggtccggcaacctccagggaagggcctggagtgggtctcaagtattaattggaatagtggtcgcatagcctatgcggactctgtgaagggccgattcaccatctccagagacaacgccaggaattccctgtatctgcaaatgaacagtctgagacttgaggacacggccttctattactgtgcaaaagatatacggaggtttagtaccgggggggcggagtttgagtactggggccagggaaccctggtcaccgtctcctca

SEQ ID NO: 37 - DNA, 390 bp, Artificial Sequence
  Description of Artificial Sequence Synthetic polynucleotide
atggccggcttccctctcctcctcaccctcctcactcactgtgcagggtcttgggcccagtctgtgctgactcagccgccctcagcgtctgggacccccgggcagagggtcaccatctcttgttctggaagcagctccaacatcggaagtaattttgtatactggtaccagcagctcccaggaacggcccccaaactcctcatatataggaataatcagcggccctcaggggtccctgaccgattctctggctccaggtctggcacctcagcctccctggccatcagtggactccggtccgaggatgaggctgattattactgtgcagcatgggatgacagcctgggaggccattatgtcttcggaactgggaccaaggtcaccgtcctt

SEQ ID NO: 38 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 39 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 40 - protein, 69 aa, Unknown
  Description of Unknown CD8 sequence
STTTKPVLRTPSPVHPTGTSQPQRPEDCRPRGSVKGTGLDFACDIYIWAPLAGICVALLLSLIITLICY

SEQ ID NO: 41 - DNA, 207 bp, Unknown
  Description of Unknown CD8 sequence
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 42 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 43 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 44 - DNA, 81 bp, Homo sapiens
ttttgggtgctggtggtggttggtggagtcctggcttgctatagcttgctagtaacagtggcctttattattttctgggtg

SEQ ID NO: 45 - protein, 27 aa, Homo sapiens
FWVLVVVGGVLACYSLLVTVAFIIFWV

SEQ ID NO: 46 - DNA, 81 bp, Homo sapiens; identical to SEQ ID NO: 44

SEQ ID NO: 47 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 48 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 49 - protein, 113 aa, Unknown
  Description of Unknown CD3zeta sequence
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 50 - DNA, 342 bp, Unknown
  Description of Unknown CD3zeta sequence
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 51 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 53 - DNA, 123 bp, Mus musculus
aatagtagaaggaacagactccttcaaagtgactacatgaacatgactccccggaggcctgggctcactcgaaagccttaccagccctacgcccctgccagagactttgcagcgtaccgcccc

SEQ ID NO: 54 - protein, 41 aa, Homo sapiens
RSKRSRLLHSDYMNMTPRRPGPTRKHYQPYAPPRDFAAYRS

SEQ ID NO: 55 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 56 - protein, 108 aa, Homo sapiens
AIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 57 - DNA, 324 bp, Homo sapiens
gcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 58 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 59 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 60 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 61 - protein, 485 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1455 bp, Artificial Sequence
  Description of Artificial Sequence Synthetic polynucleotide
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 63 - protein, 101 aa, Homo sapiens
MAMMEVQGGPSLGQTCVLIVIFTVLLQSLCVAVTYVYFTNELKQMQDKYSKSGIACFLKEDDSYWDPNDEESMNSPCWQVKWQLRQLVRKTPRMKRLWAAK